(19) United States
(12) Patent Application Publication
(10) Pub. No.: US 2010/0082072 A1
(43) Pub. Date: Apr. 1, 2010

(54) BONE ANCHORS FOR ORTHOPEDIC APPLICATIONS

(76) Inventors: Daryl R. Sybert, New Albany, OH (US); Sigurd Berven, SanFrancisco, CA (US); Randal R. Betz, Ocean City, NJ (US); Oheneba Boachie-Adjei, New York, NY (US); Scott Boden, Atlanta, GA (US); Michael F. O'Brien, Coral Gables, FL (US); Alexis P. Shelokov, Plano, TX (US); John M. Winterbottom, Howell, NJ (US)

Correspondence Address: CHOATE, HALL & STEWART LLP, TWO INTERNATIONAL PLACE, BOSTON, MA, 02110, US

(21) Appl. No.: 12/413,013

(22) Filed: Mar. 27, 2009

Related U.S. Application Data
(60) Provisional application No. 61/040,483, filed on Mar. 28, 2008.

(51) Int. Cl. A61B 17/84; A61B 17/56

(52) U.S. Cl. 606/326; 606/86 R

(57) ABSTRACT

Bone anchors and related methods for their use are described. The inventive anchor is suitable for placement in bone and for use in orthopedic surgery and dentistry. The bone anchor can be made from a bone/polymer or bone substitute/polymer composite, and can provide a firm and secure base for attaching a fastening device. The bone anchor can be used in various orthopedic and dental procedures including spinal surgery, where normal, cancellous, cortical, diseased or osteoporotic bone is present. The bone anchor can be resorbed and/or replaced with native bone tissue over a period of time. In certain embodiments, the bone anchor is made malleable or flowable and formed in situ or in vivo.

### CROSS-REFERENCE TO RELATED U.S. APPLICATIONS

The present application claims priority under 35 U.S.C. §119(e) to U.S. provisional patent application, U.S. Ser. No. 61/040,483, filed on Mar. 28, 2008, which is incorporated herein by reference.

### FIELD OF THE INVENTION

The invention pertains to implantable bone anchors useful in orthopedic surgery and dentistry. In particular, the bone anchors are made from bone/polymer composites or bone substitute/polymer composites, can be preformed prior to implantation or formed in situ, and can optionally expand upon insertion of a mechanical fastener into the anchor. The invention also provides methods of using and preparing bone anchors.

### BACKGROUND

Bone is a composite material composed of impure hydroxyapatite, collagen, and a variety of non-collagenous proteins, as well as embedded and adherent cells. Bone-derived biomaterials can be used in the preparation of osteoimplants. For example, bone particles can be combined with one or more polymers to create composites that are soft, moldable, and/or flexible under certain conditions as has been disclosed in U.S. Pat. No. 7,291,345, filed Dec. 12, 2003; and U.S. patent application Ser. No. 11/625,119, filed Jan. 19, 2007, and published under publication number 2007/0191963; each of which is incorporated herein by reference.

The use of composites in orthopedic medicine and dentistry is well known. While bone wounds can regenerate without the formation of scar tissue, fractures and other orthopedic injuries take a long time to heal, during which the injured bone is unable to support physiologic loading. Metal pins and screws are frequently placed in bone during orthopedic surgery. However, metal is significantly stiffer than bone, and in some cases the bone cannot provide a secure, firm anchoring site for a metal fastener. For example, osteoporotic bone has decreased density and may be unsuitable for anchoring metal or non-metal fasteners or other fixtures. In some cases, the use of metal implants can cause a decrease in bone density around the implant site due to stress shielding. A problem resulting from decreased bone density is pull-out of the metal fixture at the implant site. Osteoimplants useful as anchors to hold screws, pins, or other metal fasteners firmly in bone are therefore desirable.

### SUMMARY OF THE INVENTION

The present invention stems from the recognition that anchoring devices made of bone/polymer or bone substitute/polymer composites would be useful for orthopedic surgery and/or dentistry. In various embodiments, an implantable bone anchor is fabricated or molded from a bone/polymer composite, or a bone substitute/polymer composite, into any of a variety of useful shapes adapted for use at an implant or placement site in a bone, e.g., a void in a vertebra, sacrum, femur, humerus, etc. The inventive bone anchor can be adapted to receive a fastening device and provide secure and firm attachment of the fastening device to the bone at the placement site. In certain embodiments, the material from which the anchor has been prepared is solid-setting, such that it becomes load-bearing immediately after setting into a rigid or substantially solid state at the implant site. In certain embodiments, the material is moldable at the time the anchor is placed, and then later becomes set. The anchor can have expanding characteristics, such that at least a portion of the anchor expands into intimate contact with surrounding bone. For example, the anchor can mechanically expand upon insertion of a fastening device, e.g. a screw, pin, post, etc, into the anchor. The inventive bone anchor can be preformed, e.g., provided substantially in the shape of a bone anchor device suitable for placement in a void in a bone. The inventive bone anchor can be non-preformed, e.g., provided as a mass of material which can be molded or formed into a bone anchor suitable for placement in a void in a bone.

In various embodiments, the invention includes surgical methods relating to the placement of the inventive bone anchor. An embodiment of an inventive surgical method comprises evaluating an implant site, and providing the inventive bone anchor to the implant site such that the bone anchor improves the integrity of the implant site for receiving a fastening device. An embodiment of a surgical method comprises evaluating a characteristic of bone at a placement site in a subject to be treated with the bone anchor, selecting a type of bone anchor, e.g., a preformed or non-preformed bone anchor, based upon the evaluated characteristics, preparing the site to receive the bone anchor, and providing the bone anchor to the prepaired site. In certain embodiments, the placement site is located in a vertebra of the spine, e.g., in a thoracic or lumbar vertebra, or in the sacrum. In certain embodiments, the placement site is located in a pedicle or vertebral body. In various embodiments, the step of preparing the placement site comprises any combination of reaming, drilling, grinding, cutting, and threading bone at the site. In various embodiments, the inventive bone anchor is provided to the placement site in a manner to improve the integrity of bone at the placement site for receiving a fastening device, e.g., a pedicle screw, a fixation device, a screw, a pin, a rod. In some embodiments, a surgical method comprises placing an inventive bone anchor in a pedicle of a vertebra such that the pedicle/bone anchor combination receives and secures a pedicle screw. In certain embodiments, the inventive bone anchor partipates in stabilization, relocation, restructuring, revising, or immobilization of a bone.

In various embodiments, the bone anchor comprises a preformed elongate element formed from a composite and adapted for placement within a void in a bone. The anchor can have a near end, a distal end, an inner surface and outer surface and further be adapted to receive and secure a fastening device. In some embodiments, the bone anchor has engagement means, e.g., threads, ridges, grooves, barbs, barbed rings, etc., to engage with the surrounding bone. In certain embodiments, the bone anchor is adapted to engage with the surrounding bone of a pedicle, a vertebral body, or a combination thereof. In various embodiments the composite comprises a plurality of particles and a polymer with which the particles have been combined, e.g., a bone/polymer or bone substitute/polymer composite. The particles can include particles of bone-derived material, bone particles, bone substitute material, inorganic particles and any combination thereof.

In certain embodiments, the composite is capable of transitioning or transforming reversibly between different phase-states, e.g., from a substantially solid state to a malleable, moldable, pliable, or flowable state, back to a substantially solid state. In some embodiments, the composite transitions irreversibly between two phase-states, e.g., from a malleable, moldable, pliable, or flowable state to a substantially solid state. In certain embodiments, the composite is malleable under certain conditions, e.g., subjected to a high temperature or subjected to a certain solvent, and substantially rigid or solid under different conditions, e.g., subjected to a lower temperature, exposure to radiation, exposure to chemical reagent, subjected to evaporative conditions. The malleable composite can range in viscosity from a thick, flowable, or injectable liquid to a moldable, pliable, dough-like substance. In particular embodiments, phase-state transitions occur within biocompatible temperature ranges or biocompatible chemical conditions. In certain embodiments, an anchor formed from a malleable composite provides intimate contact with the irregular surfaces of the surrounding native bone.

The inventive bone anchor can be formed from a composite or material disclosed in any of the following patents or patent applications: U.S. Pat. No. 7,291,345, issued Nov. 6, 2007; U.S. Pat. No. 7,270,813, issued Sep. 18, 2007; U.S. Pat. No. 7,179,299, issued Feb. 20, 2007; U.S. Pat. No. 6,843,807, issued Jan. 18, 2005; U.S. Pat. No. 6,696,073, issued Feb. 24, 2004; U.S. Pat. No. 6,478,825, issued Nov. 12, 2002; U.S. Pat. No. 6,440,444, issued Aug. 27, 2002; U.S. Pat. No. 6,332,779, issued Dec. 25, 2001; U.S. Pat. No. 6,294,041, issued Sep. 25, 2001; U.S. Pat. No. 6,294,187, issued Sep. 25, 2001; U.S. Pat. No. 6,123,731, issued Sep. 26, 2000; U.S. Pat. No. 5,899,939, issued May 4, 1999; U.S. Pat. No. 5,507,813, issued Apr. 16, 1996; U.S. patent application, U.S. Ser. No. 10/639,912, filed Aug. 12, 2003; U.S. patent application, U.S. Ser. No. 10/736,799, filed Dec. 16, 2003; U.S. patent application, U.S. Ser. No. 10/759,904, filed Jan. 16, 2004; U.S. patent application, U.S. Ser. No. 10/771,736, filed Feb. 2, 2004; U.S. patent application, U.S. Ser. No. 11/047,992, filed Jan. 31, 2005; U.S. patent application, U.S. Ser. No. 11/336,127, filed Jan. 19, 2006; U.S. patent application, U.S. Ser. No. 11/725,329, filed Mar. 20, 2007; U.S. patent application, U.S. Ser. No. 11/698,353, filed Jan. 26, 2007; U.S. patent application, U.S. Ser. No. 11/625,086, filed Jan. 19, 2007; U.S. patent application, U.S. Ser. No. 11/625,119, filed Jan. 19, 2007; U.S. patent application, U.S. Ser. No. 11/667,090, filed Nov. 5, 2005; U.S. patent application, U.S. Ser. No. 11/758,751, filed Jun. 6, 2007; U.S. Ser. No. 11/934,980, filed Nov. 5, 2007; international PCT patent application, PCT/US03/039704, filed Dec. 12, 2003; international PCT patent application, PCT/US04/03233, filed Feb. 4, 2004; international PCT patent application, PCT/US05/015426, filed May 4, 2005; international PCT patent application, PCT/US07/001,325, filed Jan. 19, 2007; international PCT patent application, PCT/US07/01326, filed Jan. 19, 2007; and international PCT patent application, PCT/US07/001,540, filed Jan. 19, 2007. Each of these patents and patent applications is incorporated herein by reference. In various embodiments, an inventive bone anchor in accordance with the teachings herein provides a new use for a composite or material disclosed in these patents and applications.

In some embodiments, the inventive bone anchor is provided in a substantially solid state, comprising a solid composite, a solid plastic, a ceramic, a metal, or any combination thereof. A bone anchor provided in a substantially solid state can be provided as a preformed device. In certain embodiments, a preformed bone anchor can be made malleable or moldable by the addition of heat or a chemical additive. In some embodiments, the inventive bone anchor is provided in a non-preformed shape, which can be made malleable or moldable by the addition of heat or a chemical additive. When made malleable or moldable, the bone anchor can be adapted to fit into a void at a placement site and improve the integrity of bone at the placement site.

The inventive bone anchor can be formed into any of a variety of shapes. For example, bone-anchor shapes can include rods, cylinders, cones, rectangles, cubes, oval cylinders, partial cylindrical strips, tubes, polygonal tubes, and pyramids. In some embodiments, the bone anchor comprises a substantially cylindrically-shaped structure, optionally threaded on its outer surface. In some embodiments, the outer surface has grooves, ridges, ribs, protrusions, or the like which assist in holding the anchor securely at the implant site. The bone anchor can optionally contain a hollow center or core which can be threaded or without threads. In certain embodiments, the anchor comprises at least one slot permitting outward expansion of at least a portion of the anchor upon insertion of a fastening device into the anchor. In various embodiments, the bone anchor is tapered inward or outward on its outer surface, and is optionally tapered inward or outward on its inner surface. In some embodiments, the inner diameter of the anchor has at least two values along the axis of the anchor. In certain aspects, the bone anchor can be formed as pieces of a cylindrical tube, each individually implantable into a void in native bone to form in combination a bone anchor.

The inventive anchors provide screw purchase, or secure anchoring which can be gripped by screws or other types of fastening devices, into different bone types, e.g., normal bone, osteoporotic bone, cortical bone, cancellous bone, diseased bone, defective bone, deformed bone, bone which has undergone traumatic injury, bone needing revision from prior surgical intervention. The types of medical screws can include, but are not limited to, cancellous, cortical, malleolar screws as well as pedicle screws. The inventive anchors can be used for different procedures at any skeletal site in the body where normal, cancellous, diseased, deformed, injured, defective, or osteoporotic bone may be present, e.g., placing a plate over a fracture, fusing vertebrae, repairing a pedicle, revision surgery of damaged bone, repairing broken or traumatized bone, spinal surgery, etc. As an example, the anchors can be placed at a site having osteoporotic bone to improve purchase of screws which secure a plate, pins, rods or the like.

In certain aspects, the invention provides methods for making and forming a bone anchor. In some embodiments, bone particles and/or particles of a bone substitute material are combined with a polymer and mixed until the substance becomes a substantially homogeneous composite. A solvent or heat can be used during the mixing phase to aid in dispersing the particles homogeneously throughout the mixture. The composite can be rendered in or transformed to a moldable of flowable state, and the moldable or flowable composite introduced into a mold comprising the shape of an anchor. The methods of making or forming a bone anchor can include treating the bone/polymer or bone substitute/polymer composite until it becomes moldable or flowable. For example, in some embodiments the composite is heated to a temperature between approximately 40° C. and approximately 130° C. to make it moldable or flowable. In some embodiments, a solvent or pharmaceutically acceptable excipient is added to the composite to make it flowable or moldable. The flowable or moldable composite can be pressed into a mold, injected into a mold, or injected into an implantation site directly. The composite can be transformed to a solid state, after which the mold can be released from the formed bone anchor. The loss of heat, solvent, or excipient from the composite comprising the anchor can cause the implant to solidify. A fastening device can be placed in the anchor immediately after the anchor is placed, or after a specified amount of time after which the anchor is set.

In another aspect, the invention provides methods for placing an inventive bone anchor. The methods are particularly useful in orthopedic surgery and dentistry, and particularly useful in spinal surgery. In various embodiments, the methods include providing an inventive bone anchor to a patient in need thereof, and placing the inventive anchor at a placement site within the patient and subsequently securing a fastening device into the bone anchor. The placement site can comprise a void in any bone of a human or animal, e.g., a void in the pedicle and/or the body of a vertebra or the sacrum. In some embodiments, the anchor is adapted to conform to the implant site, e.g., cut to a desired length prior to or during implantation, formed to a desired size and shape prior to or during implantation. In some embodiments, the composite is injected into a void at the implantation site, and a hole is formed in the composite to receive a fastening device. In some embodiments, the composite is formed and solidified in situ or in vivo into a bone anchor. In some embodiments, the inventive bone anchor is placed by preparing a hole in bone, placing a guide wire, pin or rod in the prepared hole, and guiding the bone anchor to the prepared hole using the guide wire, pin or rod. In certain embodiments, pieces of an inventive anchor are placed in the implant site sequentially to form an anchor, and a fastening device is subsequently placed in the assembled anchor. In additional embodiments, the bone anchor is shaped according to the implant site immediately prior to implantation and placed in the implant site. A fastening device can subsequently be placed in an implanted anchor.

In certain embodiments, bone at a placement site is normal bone. In various embodiments, the bone anchor is used to treat bone having an undesirable characteristic at a placement site. The bone can be cancellous, diseased, deformed, traumatically injured, defective, osteoporotic, or any combination thereof. The bone anchor can be used to various bone disorders including genetic diseases, congenital abnormalities, fractures, iatrogenic defects, bone cancer, trauma to the bone, surgically created defects or damage to the bone which need revision, bone metastases, inflammatory diseases (e.g. rheumatoid arthritis), autoimmune diseases, metabolic diseases, and degenerative bone disease (e.g., osteoarthritis). In certain embodiments, an inventive bone anchor is formed or selected for the repair of a simple fracture, compound fracture, or non-union; as part of an external fixation device or internal fixation device; for joint reconstruction, arthrodesis, arthroplasty; for repair of the vertebral column, spinal fusion or internal vertebral fixation; for tumor surgery; for deficit filling; for discectomy; for laminectomy; for excision of spinal tumors; for an anterior cervical or thoracic operation; for the repairs of a spinal injury; for scoliosis, for lordosis or kyphosis treatment; for intermaxillary fixation of a fracture; for mentoplasty; for temporomandibular joint replacement; for alveolar ridge augmentation and reconstruction; as an inlay osteoimplant; for implant placement and revision; for revision surgery of a total joint arthroplasty; for staged reconstruction surgery; and for the repair or replacement of the cervical vertebra, thoracic vertebra, lumbar vertebra, and sacrum; and for the attachment of a screw or other component to osteoporotic bone. Additional uses for the inventive bone anchors include reinforcing an anchoring site for the attachment of components of a spinal stabilization system, providing stabilization of the spine for spinal fusion procedures, including posterior lumbar interbody fusion (PLIF), anterior lumbar interbody fusion (ALIF), transforaminal lumbar interbody fusion (TLIF), other interbody fusion procedures in the lumbar, thoracic or cervical spine, posterolateral fusion in the cervical, thoracic or lumbar spine, treatment of osteoporotic or traumatic compression fractures of the vertebrae, adult spinal deformity correction, pediatric spinal deformity correction (scoliosis), etc.

In another aspect, the invention provides various kits for use in orthopedic or dental procedures. A bone anchor kit can include at least one inventive bone anchor as described above or composite for at least one bone anchor. In some embodiments, a kit includes a tool for preparing or adapting a placement site to accommodate a bone anchor provided with the kit. The kit can further include a tool for adapting a bone anchor provided with the kit to fit into or conform to a placement site. In some embodiments, a bone anchor kit includes at least one tool or chemical reagent for changing the phase-state of the bone anchor composite. The kit can further include at least one mold of a bone anchor, a tool for placing the anchor, a tool for altering the shape of the anchor, e.g., a cutting or grinding instrument, one or more fastening devices compatible with at least one bone anchor provided by the kit, and user instructions. The inventive kit can further include a fastening-device form compatible with at least one bone anchor provided by the kit.

It will be appreciated that a variety of kits can be assembled to provide the inventive bone anchor and related tools or chemical components. Various additional examples of bone anchor kits follow. One embodiment of a kit includes at least one preformed inventive bone anchor and can optionally include instructions for placing and using the anchor. In some embodiments, a kit includes a plurality of preformed anchors in similar or various sizes and shapes, for example 2, 3, 5, 10, 15, etc. anchors per kit with anchor diameters of substantially equivalent value, or varying from about 5 millimeters to about 20 millimeters. Another embodiment of a kit includes a quantity of bone/polymer or bone substitute/polymer composite in an amount sufficient to form at least one bone anchor, optionally one or more anchor molds, and optionally include instructions for forming and using the inventive anchor. Another embodiment of a kit includes a quantity of bone/polymer or bone substitute/polymer composite in an amount sufficient to form at least one bone anchor, one or more fastening-device forms, one or more corresponding fastening devices, an injection syringe or cannula, and instructions for forming and using the inventive anchor, fastening-device form, and fastening device. Various amounts of the composite can be packaged in a kit, and all components of the kit, and the kit itself, can be sterilely packaged. The kits can further include an apparatus, reagent, solvent, or material for making the composite moldable or flowable, e.g. a heating device, solvent, or a pharmaceutically acceptable excipient. The kits can further include an apparatus, reagent, solvent, or material that will cause the composite to substantially solidify or set, e.g., a heating device, a chemical, a source of ultraviolet, infrared or microwave radiation. Any of the kits can further include one or more types of fastening devices compatible with the inventive anchors.

### DEFINITIONS

“Biomolecules”: The term “biomolecules,” as used herein, refers to classes of molecules (e.g., proteins, amino acids, peptides, polynucleotides, nucleotides, carbohydrates, sugars, lipids, nucleoproteins, glycoproteins, lipoproteins, steroids, etc.) that are commonly found in cells and tissues, whether the molecules themselves are naturally-occurring or artificially created (e.g., by synthetic or recombinant methods). For example, biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA.

“Biocompatible”: The term “biocompatible,” as used herein is intended to describe materials that, upon administration in vivo, do not induce undesirable long term effects.

“Biodegradable”: As used herein, “biodegradable” materials are materials that degrade under physiological conditions to form a product that can be metabolized or excreted without damage to organs. Biodegradable materials are not necessarily hydrolytically degradable and may require enzymatic action to fully degrade. Biodegradable materials also include materials that are broken down within cells.

“Composite”: As used herein, the term “composite” is used to refer to a unified combination of two or more distinct materials.

“Formable”: As used herein, “formable” materials are those that can be shaped by mechanical deformation. Exemplary methods of deformation include, without limitation, injection molding, extrusion, pressing, casting, rolling, and molding. In one embodiment, formable materials can be shaped by hand or using hand-held tools, much as an artist manipulates clay.

“Glass Transition Temperature”: As used herein, the term “glass transition temperature” (T_{g}) indicates the lowest temperature at which an amorphous or partially amorphous polymer is considered softened and possibly flowable. As referred to herein, the value of T_{g} is to be determined using differential calorimetry as per ASTM Standard E1356-98 “Standard Test Method for Assignment of the Glass Transition Temperatures by Differential Scanning Calorimetry or Differential Thermal Analysis.”

“Melting Temperature”: As used herein, the term “melting temperature” (Tₘ) is defined as the temperature, at atmospheric pressure, at which a polymer changes its state from solid to liquid. As referred to herein, the value of Tₘ is the value of Tₚₘ₁ as determined according to per ASTM Standard D3418-99 “Standard Test Method for Transition Temperatures of Polymers By Differential Scanning Calorimetry.”

“Osteoinductive”: As used herein, the term “osteoinductive” is used to refer to the ability of a substance to recruit cells from the host that have the potential for forming new bone and repairing bone tissue. Most osteoinductive materials can stimulate the formation of ectopic bone in soft tissue.

“Osteoconductive”: As used herein, the term “osteoconductive” is used to refer to the ability of a non-osteoinductive substance to serve as a suitable template or substrate along which bone may grow.

“Osteoimplant”: As used herein, the term “osteoimplant” does not imply that the implant contains a specific percentage of bone or has a particular shape, size, configuration or application.

“Polynucleotide,” “nucleic acid,” or “oligonucleotide”: The terms “polynucleotide,” “nucleic acid,” or “oligonucleotide” refer to a polymer of nucleotides. The terms “polynucleotide”, “nucleic acid”, and “oligonucleotide”, may be used interchangeably. Typically, a polynucleotide comprises at least three nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Polypeptide”, “peptide”, or “protein”: According to the present invention, a “polypeptide,” “peptide,” or “protein” comprises a string of at least three amino acids linked together by peptide bonds. The terms “polypeptide”, “peptide”, and “protein”, may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain; see, for example, www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

“Polysaccharide”, “carbohydrate” or “oligosaccharide”: The terms “polysaccharide,” “carbohydrate,” or “oligosaccharide” refer to a polymer of sugars. The terms “polysaccharide”, “carbohydrate”, and “oligosaccharide”, may be used interchangeably. Typically, a polysaccharide comprises at least three sugars. The polymer may include natural sugars (e.g., glucose, fructose, galactose, mannose, arabinose, ribose, and xylose) and/or modified sugars (e.g., 2′-fluororibose, 2′-deoxyribose, and hexose).

“Settable”: As used herein, the term “settable” refers to a material that can be rendered more resistant to mechanical deformation with respect to a formable state.

“Set”: As used herein, the term “set” refers to the state of a material that has been rendered more resistant to mechanical deformation with respect to a formable state.

“Small molecule”: As used herein, the term “small molecule” is used to refer to molecules, whether naturally-occurring or artificially created (e.g., via chemical synthesis), that have a relatively low molecular weight. Typically, small molecules have a molecular weight of less than about 5000 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered acceptable for use in accordance with the present invention.

“Bioactive agents”: As used herein, the term “bioactive agents” is used to refer to compounds or entities that alter, inhibit, activate, or otherwise affect biological or chemical events. For example, bioactive agents may include, but are not limited to, anti-AIDS substances, anti-cancer substances, antibiotics, immunosuppressants, anti-viral substances, enzyme inhibitors, neurotoxins, opioids, hypnotics, anti-histamines, lubricants, tranquilizers, anti-convulsants, muscle relaxants and anti-Parkinson substances, anti-spasmodics and muscle contractants including channel blockers, miotics and anti-cholinergics, anti-glaucoma compounds, anti-parasite and/or anti-protozoal compounds, modulators of cell-extracellular matrix interactions including cell growth inhibitors and anti-adhesion molecules, vasodilating agents, inhibitors of DNA, RNA, or protein synthesis, anti-hypertensives, analgesics, anti-pyretics, steroidal and non-steroidal anti-inflammatory agents, anti-angiogenic factors, anti-secretory factors, anticoagulants and/or antithrombotic agents, local anesthetics, ophthalmics, prostaglandins, anti-depressants, anti-psychotic substances, anti-emetics, and imaging agents. In a certain preferred embodiments, the bioactive agent is a drug.

A more complete listing of bioactive agents and specific drugs suitable for use in the present invention can be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996; and the United States Pharmacopeia-25/National Formulary-20, published by the United States Pharmcopeial Convention, Inc., Rockville Md., 2001, each of which is incorporated herein by reference.

The foregoing and other aspects, embodiments, and features of the present teachings can be more fully understood from the following description in conjunction with the accompanying drawings.

### BRIEF DESCRIPTION OF THE DRAWINGS

The skilled artisan will understand that the figures, described herein, are for illustration purposes only. It is to be understood that in some instances various aspects of the invention may be shown exaggerated or enlarged to facilitate an understanding of the invention. In the drawings, like reference characters generally refer to like features, functionally similar and/or structurally similar elements throughout the various figures. The drawings are not necessarily to scale, emphasis instead being placed upon illustrating the principles of the teachings. The drawings are not intended to limit the scope of the present teachings in any way.

FIG. 1A represents an elevation view of an embodiment of an inventive anchor. Slots 120 near the distal end 195 of the anchor can permit outward movement or expansion of the outer walls 110 as a mechanical fastener is inserted into the anchor's center 101. Either or both of the inner wall 150 and outer wall 155 can be threaded. FIG. 1B is a plan view of the anchor depicted in FIG. 1A, viewed from the distal end 195.

FIGS. 2A-2B depict an elevation view and plan view, viewed from the distal end, of an embodiment of an inventive anchor having threads 255 and a flanged head 202. Four expansion slots 120 are incorporated in the distal end of the anchor. A slot 212 in the head 202 can be used to torque and insert the anchor in the implantation site.

FIGS. 3A-3B depict an elevation view and plan view, viewed from the near end, of an embodiment of an inventive anchor having threads and a hexagonal head 302. The hexagonal head can be used to torque and insert the anchor in the implantation site.

FIGS. 4A-4C depict, in elevation view, various embodiments of inventive anchors. In 4A and 4B, the inner wall 450 is tapered inwards. An inserted fastening device will act to spread the distal-end walls outward. In 4B the outer wall 455 is tapered inward. In 4C, the inner wall 450 has varied diameters along the axis of the anchor, so that an inserted fastening device will slide through portions 451 and 452 and engage threads of end portion 453. Tightening the inserted fastening device would act to compress the anchor and expand the walls along portion 452 outwards.

FIG. 5 is an elevation view depicting an embodiment of a bayonet-style anchor 501 with a pin-in-rivet fastener 500. A protruding feature or pin 538 extending through the fastener 500 can slide through groove 548, engage the anchor's distal end at sloping profile 568, and lock into depression 570.

FIG. 6 is an elevation view depicting an embodiment of a latch-style anchor 601 with a flanged-rivet fastener 600.

FIG. 7 is a cross-sectional elevation view of an embodiment of a fastening-device form that can be used for forming an inventive anchor in situ.

FIG. 8 depicts a tulip-shaped inventive anchor. The anchor's distal end 895 has a flared profile, and can provide resistance against pull-out of the anchor.

FIG. 9 depicts an inventive winged anchor. Wings 970 at the anchor's distal end can provide resistance against pull-out of the anchor.

FIGS. 10A-10B depict placement of an inventive bone anchor into the pedicle of a vertebra.

The features and advantages of the present invention will become more apparent from the detailed description set forth below when taken in conjunction with the drawings.

### DETAILED DESCRIPTION OF THE EMBODIMENTS

The present invention stems from the recognition that bone at a site of surgical intervention sometimes requires supplementation to provide adequate mechanical strength or integrity to meet the needs of the surgical intervention. As an example, a pedicle of the vertebra may require supplementation to securely receive and hold a pedicle screw. Bone at the site of surgical intervention, or placement site, can be normal bone, osteoporotic bone, cortical bone, cancellous bone, diseased bone, defective bone, deformed bone, bone which has undergone traumatic injury, bone needing revision from prior surgical intervention, or any combination thereof. Generally, the bone is unable to provide adequate mechanical support, anchoring or sufficient purchase for screws, fastening devices, or other medical devices which are to be attached to the bone. In such circumstances, a formable and solid-setting implantable bone anchor or preformed bone anchor would be a useful medical device to improve the integrity of bone at the site and provide secure anchoring for a medical device to be placed at the site. Various embodiments of inventive bone anchors and related methods for their use are described.

In overview, the inventive bone anchors can be made from a composite, also referred to herein as a bone/polymer or bone substitute/polymer composite, which can be incorporated or transformed at least in part into a patient's bone after placement. In some embodiments, the composite minimally contains a polymer and another material which might be bone or a bone substitute. In certain embodiments, the inventive anchors are made from plastic, ceramic, or metal, or composites thereof. In certain embodiments, the composites are made moldable or flowable under certain conditions, and substantially solid under other conditions, e.g. heating and cooling, or in-diffusing and out-diffusing of a solvent, or addition of a catalyst, or exposure to radiation. In certain embodiments, the bone anchor is preformed prior to implantation, formed in situ, or formed in vivo, and provides a secure and firm anchor for receiving a fastening device in normal, cortical, cancellous, diseased, or osteoporotic bone, or a bony defect. A portion of the anchor can optionally expand upon insertion of a fastening device into the anchor, so as to force a portion of the anchor into intimate contact with the surrounding native bone. In various embodiments, the anchor is implanted into the pedicle of the vertebrae, or provides a patch or repair for sites where the pedicle wall has been breached. In some embodiments, the bone anchor comprises a patch or a sleeve that can be inserted into a prepared hole which has breached the cortex to cover the breach and guide a screw past the breach. The inventive anchor can be placed in the vicinity of a fracture or wound site for any bone, e.g., the mandible, femur, tarsals, ulna, radius, lumbar vertebra, sacrum, thoracic vertebra, cervical vertebra, etc. In certain embodiments, the inventive bone achors provide an attachment site for medical implants at revision in circumstances where cancellous or cortical bone may have been crushed by a previous screw placement and where the crushed cancellous or cortical bone is removable by drilling or other standard surgical means.

### Materials for Making Inventive Bone Anchors

### Bone/Polymer or Bone Substitute/Polymer Composite

In certain embodiments, a wide variety of biocompatible materials can be used to make the inventive bone anchors, e.g., plastics, polymers, ceramics, metal plastic composites, metal polymer composites, metal ceramic composites, or composites of any combination of these materials. U.S. Pat. Nos. 5,899,939; 5,507,813; 6,123,731; 6,294,041; 6,294,187; 6,332,779; 6,440,444; 6,478,825; and 7,291,345, and U.S. patent application Ser. No. 11/625,119, published under publication number 2007/0191963, each of which is incorporated herein by reference, describe various materials and methods for preparing these materials for use in orthopedic and/or dental applications. Examples of materials which can be used to make the inventive bone anchors are described below.

### Bone-Derived Material

The composite of the inventive anchor can include particles in a polymeric matrix. Any type of particles comprising inorganic material, bone substitute material, bone-derived material, or combinations or composites thereof can be utilized in the present invention to prepare the inventive bone anchors. In certain embodiments, a bone-derived material is used in the composites used to make the bone anchors. In one embodiment, bone-derived material employed in the preparation of the composite are obtained from cortical, cancellous, and/or corticocancellous bone. The bone-derived material can be derived from any vertebrate. The bone-derived material can be of autogenous, allogeneic, and/or xenogeneic origin. In certain embodiments, the bone-derived material is autogenous, that is, the bone-derived material is from the subject being treated. In other embodiments, the bone-derived material is allogeneic (e.g., from donors). Preferably, the source of the bone is matched to the eventual recipient of the inventive bone anchor (i.e., the donor and recipient are preferably of the same species). For example, human bone-derived material is typically used for bone anchors placed in a human subject. In certain particular embodiments, the bone particles are obtained from cortical bone of allogeneic origin. In certain embodiments, the bone-derived material is obtained from bone of xenogeneic origin. Porcine and bovine bone are particularly advantageous types of xenogeneic bone tissue that can be used individually or in combination as sources for the bone-derived material. Xenogeneic bone tissue can be combined with allogeneic or autogenous bone tissue.

Particles of bone-derived material are formed by any process known to break down bone into small pieces. Exemplary processes for forming such particles include milling whole bone to produce fibers, chipping whole bone, cutting whole bone, grinding whole bone, fracturing whole bone in liquid nitrogen, or otherwise disintegrating the bone tissue. Particles can optionally be sieved to produce particles of a specific size range. The particles can be of any shape or size. Exemplary shapes include spheroidal, plates, fibers, cuboidal, sheets, rods, oval, strings, elongated particles, wedges, discs, rectangular, polyhedral, etc. In some embodiments, particles are between about 10 microns and about 1000 microns in diameter or more. In some embodiments, particles are between about 20 microns and about 800 microns in diameter or more. In certain embodiments, the particles range in size from approximately 100 microns in diameter to approximately 500 microns in diameter. In certain embodiments, the particles range in size from approximately 300 microns in diameter to approximately 800 microns in diameter. As for irregularly shaped particles, the recited dimension ranges may represent the length of the greatest or smallest dimension of the particle. As will be appreciated by one of skill in the art, for injectable composites, the maximum particle size will depend in part on the size of the cannula or needle through which the material will be delivered. In some embodiments, the maximum particle size will be less than about one-quarter the size of the inner diameter of the cannula or needle through which the composite will be delivered. In some embodiments, the maximum particle size will be less than about one-tenth the size of the inner diameter of the cannula or needle through which the composite will be delivered.

In certain embodiments, the particles that are combined with a polymer to form the composite for the inventive bone anchor have a particle size distribution with respect to a mean value plus or minus a percentage value, e.g., about ±10% or less of the mean value, about ±20% or less of the mean value, about ±30% or less of the mean value, about ±40% or less of the mean value, about ±50% or less of the mean value, about ±60% or less of the mean value, about ±70% or less of the mean value, about ±80% or less of the mean value, or about ±90% or less of the mean value. In other embodiments, the particle size distribution with respect to a median value can be plus or minus a percentage value about the median value, e.g., about ±10% or less of the median value, about ±20% or less of the median value, about ±30% or less of the median value, about ±40% or less of the median value, about ±50% or less of the median value, about ±60% or less of the median value, about ±70% or less of the median value, about ±80% or less of the median value, or about ±90% or less of the median value. In certain embodiments, at least about 60, 70, or 80 weight percent of the particles posses a median length of about 10 microns to about 1000 microns in their greatest dimension. In certain embodiments, at least about 60, 70, or 80 weight percent of the particles posses a median length of about 20 microns to about 800 microns in their greatest dimension. For particles that are fibers or other elongated particles, at least about 60 weight percent, at least about 70 weight percent, or at least about 80 weight percent of the particles possess a median length of from about 2 to about 200 mm, or more preferably from about 10 to about 100 mm, a median thickness of from about 0.05 to about 2 mm, and preferably from about 0.2 to about 1 mm, and a median width of from about 1 mm to about 20 mm and preferably from about 2 to about 5 mm. The particles can possess a median length to median thickness ratio from at least about 5:1 up to about 500:1, preferably from at least about 50:1 up to about 500:1, or more and preferably from about 50:1 up to about 100:1; and a median length to median width ratio of from about 10:1 to about 200:1 and preferably from about 50:1 to about 100:1. In certain embodiments, the bone-derived particles are short fibers having a cross-section of about 300 microns to about 100 microns and a length of about 1 mm to about 4 mm.

The processing of the bone to provide the particles can be adjusted to optimize for the desired size and/or distribution of the particles. The desired properties of the resulting bone anchor (e.g., mechanical properties) can also be engineered by adjusting the weight percent, shapes, sizes, distribution, etc. of the bone-derived particles or other particles. For example, the composite can be made more viscous by including a higher percentage of particles.

The bone-derived particles utilized in accordance with the present invention can be demineralized, non-demineralized, mineralized, or anorganic. In certain embodiments, the resulting bone-derived particles are used “as is” in preparing the composite used in making the inventive bone anchor. In other embodiments, the particles are defatted and disinfected. An exemplary defatting/disinfectant solution is an aqueous solution of ethanol. Other organic solvent can also be used in the defatting and disinfecting the particles. For example, methanol, isopropanol, butanol, DMF, DMSO, diethyl ether, hexanes, glyme, tetrahydrofuran, chloroform, methylene chloride, and carbon tetrachloride can be used. In certain embodiments, a non-halogenated solvent is used. The defatting/disinfecant solution can also include a detergent (e.g., an aqueous solution of a detergent). Ordinarily, at least about 10 to about 40 percent by weight of water (i.e., about 60 to about 90 weight percent of defatting agent such as alcohol) should be present in the defatting/disinfecting solution to produce optimal lipid removal and disinfection within the shortest period of time. An exemplary concentration range of the defatting solution is from about 60 to about 85 weight percent alcohol, for example, about 70 weight percent alcohol.

In certain embodiments, at least a portion of the particles used to make the composite for the inventive bone anchor are demineralized. The bone-derived particles are optionally demineralized in accordance with known and/or conventional procedures in order to reduce their inorganic mineral content. Demineralization methods remove the inorganic mineral component of bone by employing acid solutions. Such methods are well known in the art, see for example, Reddi, et al., Proc. Nat. Acad. Sci., 1972, 69:1601-1605, the contents of which are incorporated herein by reference. The strength of the acid solution, the shape and dimensions of the bone-derived particles, and the duration of the demineralization treatment will determine the extent of demineralization. Reference in this regard is made to Lewandrowski, et al., J. Biomed. Mater. Res., 1996, 31:365-372 and U.S. Pat. No. 5,290,558, the contents of both of which are incorporated herein by reference.

In an exemplary defatting/disinfecting/demineralization procedure, the bone-derived particles are subjected to a defatting/disinfecting step, followed by an acid demineralization step. An exemplary defatting/disinfectant solution is an aqueous solution of ethanol. Ordinarily, at least about 10 to about 40 percent by weight of water (i.e., about 60 to about 90 weight percent of defatting agent such as alcohol) should be present in the defatting/disinfecting solution to produce optimal lipid removal and disinfection within a reasonable period of time. An exemplary concentration range of the defatting solution is from about 60 to about 85 weight percent alcohol, for example, about 70 weight percent alcohol. Ethanol is typically the alcohol used in this step; however, other alcohols such as methanol, propanol, isopropanol, denatured ethanol, etc. can also be used. Following defatting, the bone particles are immersed in acid over time to effect their demineralization. The acid also disinfects the bone by killing viruses, vegetative microorganisms, and spores. Acids which can be employed in this step include inorganic acids such as hydrochloric acid and organic acids such as peracetic acid. After acid treatment, the demineralized bone particles are rinsed with sterile water to remove residual amounts of acid and thereby raise the pH. The bone particles can be dried, for example, by lyophilization, before being incorporated into a composite used to make the bone anchor. The bone particles can be stored under aseptic conditions, for example, in a lyophilized state, until they are used or sterilized using known methods (e.g., gamma irradiation) shortly before combining them with a polymer.

As utilized herein, the phrase “superficially demineralized” as applied to the bone particles refers to bone particles possessing at least about 90% by weight of their original inorganic mineral content. The phrase “partially demineralized” as applied to the bone particles refers to bone particles possessing from about 8% to about 90% weight of their original inorganic mineral content, and the phrase “fully demineralized” as applied to the bone particles refers to bone particles possessing less than about 8%, preferably less than about 1%, by weight of their original inorganic mineral content. The unmodified term “demineralized” as applied to the bone particles is intended to cover any one or combination of the foregoing types of demineralized bone particles, that is, superficially demineralized, partially demineralized, or fully demineralized bone particles.

In an alternative embodiment, surfaces of bone particles are lightly demineralized according to the procedures in U.S. patent application Ser. No. 10/285,715, filed Nov. 1, 2002, published as U.S. Patent Publication No. 2003/0144743, on Jul. 31, 2003, now U.S. Pat. No. 7,179,299, issued Feb. 20, 2007, the contents of which are incorporated herein by reference. Even minimal demineralization, for example, of less than 5% removal of the inorganic phase, increases the hydroxylation of bone fibers and the surface concentration of amine groups. Demineralization can be so minimal, for example, less than 1%, that the removal of the calcium phosphate phase is almost undetectable. Rather, the enhanced surface concentration of reactive groups defines the extent of demineralization. This can be measured, for example, by titrating the reactive groups. In one embodiment, in a polymerization reaction that utilizes the exposed allograft surfaces to initiate a reaction, the amount of unreacted monomer remaining is used to estimate reactivity of the surfaces. Surface reactivity can be assessed by a surrogate mechanical test, such as a peel test of a treated coupon of bone adhering to a polymer.

In certain embodiments, the bone-derived particles are subjected to a process that partially or totally removes their initial organic content to yield mineralized and anorganic bone particles, respectively. Different mineralization methods have been developed and are known in the are (Hurley et al., Milit. Med. 1957, 101-104; Kershaw, Pharm. J. 6:537, 1963; and U.S. Pat. No. 4,882,149; each of which is incorporated herein by reference). For example, a mineralization procedure can include a de-greasing step followed by a basic treatment (with ammonia or another amine) to degrade residual proteins and a water washing (U.S. Pat. Nos. 5,417,975 and 5,573,771; both of which are incorporated herein by reference). Another example of a mineralization procedure includes a defatting step where bone particles are sonicated in 70% ethanol for 1-3 hours.

If desired, the bone-derived particles can be modified in one or more ways, e.g., their protein content can be augmented or modified as described, for example, in U.S. Pat. Nos. 4,743,259 and 4,902,296, the contents of both of which are incorporated herein by reference.

Mixtures or combinations of one or more of the foregoing types of bone-derived particles can be employed in the composite used to prepare the inventive bone anchors. For example, one or more of the foregoing types of demineralized bone-derived particles can be employed in combination with non-demineralized bone-derived particles, i.e., bone-derived particles that have not been subjected to a demineralization process, or inorganic materials. The amount of each individual type of bone-derived particle employed can vary widely depending on the mechanical and biological properties desired. Thus, mixtures of bone-derived particles of various shapes, sizes, and/or degrees of demineralization can be assembled based on the desired mechanical, thermal, chemical, and biological properties of the composite. A desired balance between the various properties of the composite bone anchor (e.g., a balance between mechanical and biological properties) can be achieved by using different combinations of particles. Suitable amounts of various particle types can be readily determined by those skilled in the art on a case-by-case basis by routine experimentation.

The differential in strength, osteogenicity, and other properties between partially and fully demineralized bone-derived particles on the one hand, and non-demineralized, superficially demineralized bone-derived particles, inorganic ceramics, and bone substitutes on the other hand can be exploited. For example, in order to increase the compressive strength of an implant, the ratio of nondemineralized and/or superficially demineralized bone-derived particles to partially or fully demineralized bone-derived particles can be increased, and vice versa. The bone-derived particles in the composite also play a biological role. Non-demineralized bone-derived particles bring about new bone in-growth by osteoconduction. Demineralized bone-derived particles likewise play a biological role in bringing about new bone in-growth by osteoinduction. Both types of bone-derived particles are gradually remodeled and replaced by new host bone as degradation of the composite progresses over time. Thus, the use of various types of bone particles can be used to control the overall mechanical and biological properties, e.g., the strength, osteoconductivity, and/or osteoinductivity, etc., of the bone anchor.

### Surface Modification of Bone-Derived Particles

The bone-derived particles can be optionally treated to enhance their interaction with the polymer of the composite or to confer some property to the particle surface. While some bone-derived particles can interact readily with a monomer and be covalently linked to the polymer matrix, it may be desirable to modify the surface of the bone-derived particles to facilitate incorporation into polymers that do not bond well to bone, such as poly(lactides). Surface modification can provide a chemical substance that is strongly bonded to the surface of the bone, e.g., covalently bonded to the surface. The bone-derived particles can also be coated with a material to facilitate interaction with the polymer of the composite, from which the inventive bone anchor is formed.

In one embodiment, silane coupling agents are employed to link a monomer or initiator molecule to the surface of the bone-derived particles. The silane has at least two sections, a set of three leaving groups and an active group. The active group can be connected to the silicon atom in the silane by an elongated tether group. An exemplary silane coupling agent is 3-trimethoxysilylpropylmethacrylate, available from Union Carbide. The three methoxy groups are the leaving groups, and the methacrylate active group is connected to the silicon atom by a propyl tether group. In one embodiment, the leaving group is an alkoxy group such as methoxy or ethoxy. Depending on the solvent used to link the coupling agent to the bone-derived particle, hydrogen or alkyl groups such as methyl or ethyl can serve as the leaving group. The length of the tether determines the intimacy of the connection between the polymer matrix and the bone-derived particle. By providing a spacer between the bone-derived particle and the active group, the tether also reduces competition between chemical groups at the particle surface and the active group and makes the active group more accessible to the monomer during polymerization.

In one embodiment, the active group is an analog of the monomer of the polymer used in the composite. For example, amine active groups will be incorporated into polyamides, polyesters, polyurethanes, polycarbonates, polycaprolactone, and other polymer classes based on monomers that react with amines, even if the polymer does not contain an amine. Hydroxy-terminated silanes will be incorporated into polyamino acids, polyesters, polycaprolactone, polycarbonates, polyurethanes, and other polymer classes that include hydroxylated monomers. Aromatic active groups or active groups with double bonds will be incorporated into vinyl polymers and other polymers that grow by radical polymerization (e.g., polyacrylates, polymethacrylates). It is not necessary that the active group be monofunctional. Indeed, it may be preferable that active groups that are to be incorporated into polymers via step polymerization be difunctional. A silane having two amines, even if one is a secondary amine, will not terminate a polymer chain but can react with ends of two different polymer chains. Alternatively, the active group can be branched to provide two reactive groups in the primary position.

An exemplary list of silanes that can be used with the composite is provided in U.S. Patent Publication No. 2004/0146543, the contents of which are incorporated herein by reference. Silanes are available from companies such as Union Carbide, AP Resources Co. (Seoul, South Korea), and BASF. Where the silane contains a potentially non-biocompatible moiety as the active group, it should be used to tether a biocompatible compound to the bone particle using a reaction in which the non-biocompatible moiety is the leaving group. It may be desirable to attach the biocompatible compound to the silane before attaching the silane to the bone-derived particle, regardless of whether the silane is biocompatible or not. The derivatized silanes can be mixed with silanes that can be incorporated directly into the polymer and reacted with the bone-derived particles, coating the bone particles with a mixture of “bioactive” silanes and “monomer” silanes. U.S. Pat. No. 6,399,693, the contents of which are incorporated herein by reference discloses composites of silane modified polyaromatic polymers and bone. Silane-derivatized polymers can be used in the composite used to make the bone anchor instead of or in addition to first silanizing the bone-derived particles.

The active group of the silane can be incorporated directly into the polymer or can be used to attach a second chemical group to the bone particle. For example, if a particular monomer polymerizes through a functional group that is not commercially available as a silane, the monomer can be attached to the active group.

Non-silane linkers can also be employed to produce composites useful for making the inventive bone anchor. For example, isocyanates will form covalent bonds with hydroxyl groups on the surface of hydroxyapatite ceramics (de Wijn, et al., “Grafting PMMA on Hydroxyapatite Powder Particles using Isocyanatoethylmethacrylate,” Fifth World Biomaterials Congress, May 29-Jun. 2, 1996, Toronto, Calif.). Isocyanate anchors, with tethers and active groups similar to those described with respect to silanes, can be used to attach monomer-analogs to the bone particles or to attach chemical groups that will link covalently or non-covalently with a polymer side group. Polyamines, organic compounds containing one or more primary, secondary, or tertiary amines, will also bind with both the bone particle surface and many monomer and polymer side groups. Polyamines and isocyanates may be obtained from Aldrich.

Alternatively, a biologically active compound such as a biomolecule, a small molecule, or a bioactive agent can be attached to the bone-derived particle through the linker. For example, mercaptosilanes will react with the sulfur atoms in proteins to attach them to the bone-derived particle. Aminated, hydroxylated, and carboxylated silanes will react with a wide variety functional groups. Of course, the linker can be optimized for the compound being attached to the bone-derived particle.

Biologically active molecules can modify non-mechanical properties of the composite bone anchor as it is degraded or resorbed. For example, immobilization of a drug on the bone particle allows it to be gradually released at an implant site as the bone anchor is degraded. Anti-inflammatory agents embedded within the composite will control the inflammatory response long after the initial response to placement of the anchor. For example, if a piece of the anchor fractures several weeks after placement, immobilized compounds will reduce the intensity of any inflammatory response, and the anchor will continue to degrade through hydrolytic or physiological processes. Compounds can also be immobilized on the bone-derived particles that are designed to elicit a particular metabolic response or to attract cells to the implantation site.

Some biomolecules, small molecules, and bioactive agents can also be incorporated into the polymer used in the composite. For example, many amino acids have reactive side chains. The phenol group on tyrosine has been exploited to form polycarbonates, polyarylates, and polyiminocarbonates (see Pulapura, et al., “Tyrosine-derived polycarbonates: Backbone-modified “pseudo”-poly(amino acids) designed for biomedical applications,” Biopolymers, 1992, 32: 411-417; and Hooper, et al., “Diphenolic monomers derived from the natural amino acid α-L-tyrosine: an evaluation of peptide coupling techniques,” J. Bioactive and Compatible Polymers, 1995, 10:327-340, the entire contents of both of which are incorporated herein by reference). Amino acids such as lysine, arginine, hydroxylysine, proline, and hydroxyproline also have reactive groups and are essentially tri-functional. Amino acids such as valine, which has an isopropyl side chain, are still difunctional. Such amino acids can be attached to the silane and still leave one or two active groups available for incorporation into a polymer.

Non-biologically active materials can also be attached to the bone particles. For example, radioopaque, luminescent, or magnetically active particles can be attached to the bone particles using the techniques described above. If a material, for example, a metal atom or cluster, cannot be produced as a silane or other group that reacts with calcium phosphate ceramics, then a chelating agent can be immobilized on the bone particle surface and allowed to form a chelate with the atom or cluster. As the bone is resorbed, these non-biodegradable materials are still removed from the tissue site by natural metabolic processes, allowing the degradation of the polymer and the resorption of the bone-derived particles to be tracked using standard medical diagnostic techniques. The term “resorbed” is used herein to denote a transformation of at least a portion of the inventive bone anchor to host tissue.

In an alternative embodiment, the bone-derived particle surface is chemically treated before being derivatized or combined with a polymer. For example, non-demineralized bone-derived particles can be rinsed with phosphoric acid, e.g., for 1 to 15 minutes in a 5-50% solution by volume. Those skilled in the art will recognize that the relative volume of bone particles and phosphoric acid solution (or any other solution used to treat the bone particles), can be optimized depending on the desired level of surface treatment. Agitation will also increase the uniformity of the treatment both along individual particles and across an entire sample of particles. The phosphoric acid solution reacts with the mineral component of the bone to coat the particles with calcium phosphate, which can increase the affinity of the surface for inorganic coupling agents such as silanes and for the polymer component of the composite. As noted above, the surface can be partially demineralized to expose the collagen fibers at the particle surface.

The collagen fibers exposed by demineralization are typically relatively inert but have some exposed amino acid residues that can participate in reactions. The collagen can be rendered more reactive by fraying the triple helical structure of the collagen to increase the exposed surface area and the number of exposed amino acid residues. This not only increases the surface area available for chemical reactions but also for mechanical interaction with the polymer as well. Rinsing the partially demineralized bone particles in an alkaline solution will fray the collagen fibrils. For example, bone particles can be suspended in water at a pH of about 10 for about 8 hours, after which the solution is neutralized. One skilled in the art will recognize that this time period can be increased or decreased to adjust the extent of fraying. Agitation, for example, in an ultrasonic bath, may reduce the processing time. Alternatively, the particles can be sonicated with water, surfactant, alcohol, or some combination of these.

Alternatively, the collagen fibers can be cross-linked. A variety of cross-linking techniques suitable for medical applications are well known in the art (see, for example, U.S. Pat. No. 6,123,731, the contents of which are incorporated herein by reference). For example, compounds like 1-ethyl-3-(3-dimethylaminopropyl) carbodiimide hydrochloride, either alone or in combination with N-hydroxysuccinimide (NHS) will crosslink collagen at physiologic or slightly acidic pH (e.g., in pH 5.4 MES buffer). Acyl azides and genipin, a naturally occurring bicyclic compound including both carboxylate and hydroxyl groups, can also be used to cross-link collagen chains (see Simmons, et al, “Evaluation of collagen cross-linking techniques for the stabilization of tissue matrices,” Biotechnol. Appl. Biochem., 1993, 17:23-29; PCT Publication WO98/19718, the contents of both of which are incorporated herein by reference). Alternatively, hydroxymethyl phosphine groups on collagen can be reacted with the primary and secondary amines on neighboring chains (see U.S. Pat. No. 5,948,386, the entire contents of which are incorporated herein by reference). Standard cross-linking agents such as mono- and dialdehydes, polyepoxy compounds, tanning agents including polyvalent metallic oxides, organic tannins, and other plant derived phenolic oxides, chemicals for esterification or carboxyl groups followed by reaction with hydrazide to form activated acyl azide groups, dicyclohexyl carbodiimide and its derivatives and other heterobifunctional crosslinking agents, hexamethylene diisocyanate, and sugars can also be used to cross-link the collagen. The bone-derived particles are then washed to remove all leachable traces of the material. Enzymatic cross-linking agents can also be used. Additional cross-linking methods include chemical reaction, irradiation, application of heat, dehydrothermal treatment, enzymatic treatment, etc. One skilled in the art will easily be able to determine the optimal concentrations of cross-linking agents and incubation times for the desired degree of cross-linking.

Both frayed and unfrayed collagen fibers can be derivatized with monomer, pre-polymer, oligomer, polymer, initiator, and/or biologically active or inactive compounds, including but not limited to biomolecules, bioactive agents, small molecules, inorganic materials, minerals, through reactive amino acids on the collagen fiber such as lysine, arginine, hydroxylysine, proline, and hydroxyproline. Monomers that link via step polymerization can react with these amino acids via the same reactions through which they polymerize. Vinyl monomers and other monomers that polymerize by chain polymerization can react with these amino acids via their reactive pendant groups, leaving the vinyl group free to polymerize. Alternatively, or in addition, bone-derived particles can be treated to induce calcium phosphate deposition and crystal formation on exposed collagen fibers. Calcium ions can be chelated by chemical moieties of the collagen fibers, and/or calcium ions can bind to the surface of the collagen fibers. James et al., Biomaterials 20:2203-2313, 1999; incorporated herein by reference. The calcium ions bound to the to the collagen provides a biocompatible surface, which allows for the attachment of cells as well as crystal growth. The polymer will interact with these fibers, increasing interfacial area and improving the wet strength of the composite.

Additionally or alternatively, the surface treatments described above or treatments such as etching can be used to increase the surface area or surface roughness of the bone-derived particles. Such treatments increase the interfacial strength of the particle/polymer interface by increasing the surface area of the interface and/or the mechanical interlocking of the bone-derived particles and the polymer. Such surface treatments can also be employed to round the shape or smooth the edges of bone particles to facilitate delivery of the composite, e.g., when injected into a mold or implant site to form an anchor in situ.

In some embodiments, surface treatments of the bone-derived particles are optimized to enhance covalent attractions between the bone-derived particles and the polymer of the composite. In an alternative embodiment, the surface treatment can be designed to enhance non-covalent interactions between the bone-derived particle and the polymer matrix. Exemplary non-covalent interactions include electrostatic interactions, hydrogen bonding, pi-bond interactions, hydrophobic interactions, van der Waals interactions, and mechanical interlocking. For example, if a protein or a polysaccharide is immobilized on the bone-derived particle, the chains of the polymer will become physically entangled with the long chains of the biological polymer when they are combined. Charged phosphate sites on the surface of the particles, produced by washing the bone particles in basic solution, will interact with the amino groups present in many biocompatible polymers, especially those based on amino acids. The pi-orbitals on aromatic groups immobilized on a bone-derived particle will interact with double bonds and aromatic groups of the polymer.

### Bone-Substitute Materials

Inorganic materials, including, but not limited, calcium phosphate materials and bone substitute materials, can also be used as particulate inclusions in composites used to prepare the inventive anchors. Exemplary inorganics for use with the invention include aragonite, dahlite, calcite, amorphous calcium carbonate, vaterite, weddellite, whewellite, struvite, urate, ferrihydrite, francolite, monohydrocalcite, magnetite, goethite, dentin, calcium carbonate, calcium sulfate, calcium phosphosilicate, sodium phosphate, calcium aluminate, calcium phosphate, hydroxyapatite, dicalcium phosphate, α-tricalcium phosphate, β-tricalcium phosphate, tetracalcium phosphate, amorphous calcium phosphate, octacalcium phosphate, and BIOGLASS™, a calcium phosphate silica glass available from U.S. Biomaterials Corporation. Substituted calcium phosphate phases are also contemplated for use with the invention, including but not limited to fluorapatite, chlorapatite, magnesium-substituted tricalcium phosphate, and carbonate hydroxyapatite. In certain embodiments, the inorganic material is a substituted form of hydroxyapatite. For example, the hydroxyapatite can be substituted with other ions such as fluoride, chloride, magnesium, sodium, potassium, etc. Additional calcium phosphate phases suitable for use with the invention include those disclosed in U.S. Pat. Nos. RE 33,161 and RE 33,221 to Brown et al.; 4,880,610; 5,034,059; 5,047,031; 5,053,212; 5,129,905; 5,336,264; and 6,002,065 to Constantz et al.; 5,149,368; 5,262,166 and 5,462,722 to Liu et al.; 5,525,148 and 5,542,973 to Chow et al., 5,717,006 and 6,001,394 to Daculsi et al., 5,605,713 to Boltong et al., 5,650,176 to Lee et al., and 6,206,957 to Driessens et al., and biologically-derived or biomimetic materials such as those identified in Lowenstam H A, Weiner S, On Biomineralization, Oxford University Press, 1989; each of which is incorporated herein by reference.

In another embodiment, a particulate composite material is employed in the mixture with the polymer. For example, inorganic materials such as those described above or bone-derived materials can be combined with proteins such as bovine serum albumin (BSA), collagen, or other extracellular matrix components to form a composite. Alternatively or in addition, bone substitute materials or bone-derived materials can be combined with synthetic or natural polymers to form a composite using the techniques described in our co-pending U.S. Pat. No. 7,291,345, issued Nov. 6, 2007; U.S. Pat. No. 7,270,813, issued Sep. 18, 2007; and U.S. Ser. No. 10/639,912, filed Aug. 12, 2003, published as 20040146543, the contents of all of which are incorporated herein by reference. These composites can be partially demineralized as described herein to expose the organic material at the surface of the composite before they are combined with a polymer.

In certain embodiments, a particular composite useful for making the inventive bone anchors is disclosed in U.S. patent applications, U.S. Ser. No. 10/771,736, filed Feb. 2, 2004, and published as US 2005/0027033; and U.S. Ser. No. 11/336,127, filed Jan. 19, 2006, and published as US 2006/0216323; and U.S. Pat. No. 7,264,823, issued Sep. 4, 2007; and U.S. Ser. No. 10/759,904 filed Jan. 16, 2004, and published as US 2005/0013793; and U.S. Ser. No. 11/725,329 filed Mar. 20, 2007, and published as 2007/0160569; and U.S. Ser. No. 11/698,353 filed Jan. 26, 2007, and published as 2007/0190229; and U.S. Ser. No. 11/667,090 filed Nov. 5, 2005, and published as 2007/0299151, each of which is incorporated herein by reference. Composite materials described in these applications include a polyurethane matrix and a reinforcement embedded in the matrix. The polyurethane matrix can be formed by reaction of a polyisocyanate (e.g., lysine diisocyanate, toluene diisocyanate, arginine diisocyanate, asparagine diisocyanate, glutamine diisocyanate, hexamethylene diisocyanate, hexane diisocyanate, methylene bis-p-phenyl diisocyanate, isocyanurate polyisocyanates, 1,4-butane diisocyanate, uretdione polyisocyanate, or aliphatic, alicyclic, or aromatic polyisocyanates) with an optionally hydroxylated biomolecule (e.g., a phospholipids, fatty acid, cholesterol, polysaccharide, starch, or a combination or modified form of any of the above) to form a biodegradable polymer, while the reinforcement comprises bone-derived material or a bone substitute material (e.g., calcium carbonate, calcium sulfate, calcium phosphosilicate, sodium phosphate, calcium aluminate, calcium phosphate, calcium carbonate, hydroxyapatite, demineralized bone, mineralized bone, or combinations or modified forms of any of these).

Particles of composite material for use in the present invention can contain between about 5% and about 80% of bone-derived or bone substitute material, for example, between about 60% and about 75%. Particulate materials for use in the composites used to make the inventive bone anchors can be modified to increase the concentration of nucleophilic groups (e.g., amino or hydroxyl groups) at their surfaces using the techniques described herein.

Composites used to make the inventive bone anchors can contain between about 5% and 80% by weight bone-derived particles, or particles of bone substitute material. In certain embodiments, the particles make up between about 10% and about 30% by weight of the composite. In certain embodiments, the particles make up between about 30% and about 50% by weight of the composite. In certain embodiments, the particles make up between about 40% and about 50% by weight of the composite. In certain embodiments, the particles make up between about 60% and about 75% by weight of the composite. In certain embodiments, the particles make up between about 45% and about 70% by weight of the composite. In certain embodiments, the particles make up between about 50% and about 65% by weight of the composite. In certain particular embodiments, the particles make up approximately 20%, 25%, 30%, or 40% by weight of the composite. In certain particular embodiments, the particles make up approximately 45%, 46%, 47%, 48%, 49%, 50%, 51%, 52%, 53%, 54%, 55%, 56%, 57%, 58%, 59%, 60%, 61%, 62%, 63%, 64%, or 65% by weight of the composite.

Combining the Particles with a Polymer

To form a composite useful in preparing the bone anchor, the particles as discussed herein are combined with a polymer. In various embodiments, the composite is capable of undergoing at least one phase-state transition. For example, the composite can be reversibly changed from a flowable state to a moldable state to a substantially solid state, or vice versa. In some embodiments, the composite can be reversibly changed between two states, e.g. between flowable and substantially solid, between moldable and substantially solid. In certain embodiments, the composite is naturally moldable or flowable, or can be made moldable or flowable. In certain embodiments, the composite is naturally solid or semisolid and can be made moldable or flowable. The composite can be modified by cross-linking or polymerization after combination with particles to form a composite in which the polymer is covalently linked to the particles. In some embodiments, the polymer is a polymer/solvent mixture that hardens when the solvent is removed (e.g., when the solvent is allowed to evaporate or diffuse away). Exemplary solvents include but are not limited to alcohols (e.g., methanol, ethanol, propanol, butanol, hexanol, etc.), water, saline, DMF, DMSO, glycerol, and PEG. In certain embodiments, the solvent is a biological fluid such as blood, plasma, serum, marrow, lymph, extra-cellular fluid, etc. In certain embodiments, the composite used for making the inventive bone anchor is heated above the melting or glass transition temperature of one or more of its components and becomes set after implantation as it cools. In certain embodiments, the composite is set by exposing it to a heat source, or irradiating it with microwaves, IR rays, or UV light. The particles can also be mixed with a polymer that is sufficiently pliable for combining with the particles, but that may require further treatment, for example, combination with a solvent or heating, to become a flowable or moldable composite.

In some embodiments, the anchor is produced with a flowable composite and then solified or set in situ. For example, the cross-link density of a low molecular weight polymer can be increased by exposing it to electromagnetic radiation (e.g., ultraviolet (UV) light, infrared (IR) light, microwaves) or an alternative energy source. Alternatively, a photoactive cross-linking agent, chemical cross-linking agent, additional monomer, or combinations thereof can be mixed into the composite. Exposure to UV light after the composite anchor is placed at the implant site can increase one or both of the molecular weight and cross-link density, stiffening the polymer and thereby the anchor. The polymer component of the composite can also be softened by a solvent, e.g., ethanol. If a biocompatible solvent is used, the polymer can be hardened in situ. As the composite sets, solvent leaving the anchor is preferably released into the surrounding tissue without causing undesirable side effects such as irritation or an inflammatory response.

The polymer and the particulate phase can be combined by any method known to those skilled in the art. For example, a homogenous mixture of a polymer or polymer precursor and particles can be pressed together at ambient or elevated temperatures. At elevated temperatures, the process may also be accomplished without pressure. In some embodiments, the polymer or precursor is not held at a temperature of greater than approximately 60° C. for a significant time during mixing to prevent thermal damage to any biological component of the composite (e.g., bone-derived factors or cells). Alternatively or in addition, particles can be mixed or folded into a polymer softened by heat or a solvent. Alternatively, a formable polymer can be formed into a sheet that is then covered with a layer of particles. The particles can then be forced into the polymer sheet using pressure. In another embodiment, particles are individually coated with a polymer or polymer precursor, for example, using a tumbler, spray coater, or a fluidized bed, before being mixed with a larger quantity of polymer. This facilitates even coating of the particles and improves integration of the particles and polymer component of the composite.

Polymer processing techniques can also be used to combine the particles and a polymer or polymer precursor. For example, the polymer can be rendered formable, e.g., by heating or by in-diffusing with a solvent, and combined with the particles by injection molding or extrusion forming. Alternatively, the polymer and particles can be mixed in a solvent and cast with or without pressure. The composite can be prepared from both formable and rigid polymers. For example, extrusion forming can be performed using pressure to manipulate a formable or rigid polymer.

In another embodiment, the particles are mixed with a polymer precursor according to standard composite processing techniques. For example, regularly shaped particles can simply be suspended in a monomer. A polymer precursor can be mechanically stirred to distribute the particles or bubbled with a gas, preferably one that is oxygen- and moisture-free. Once the composite is mixed, it can be desirable to store it in a container that imparts a static pressure to prevent separation of the particles and the polymer precursor, which may have different densities. In some embodiments, the distribution and particle/polymer ratio are optimized to produce at least one continuous path through the composite along the particles.

The interaction of the polymer component of the composite with the particles can also be enhanced by coating individual particles with a polymer precursor before combining them with bulk precursor. The coating enhances the association of the polymer component of the composite with the particles. For example, individual particles can be spray coated with a monomer or prepolymer. Alternatively, the individual particles can be coated using a tumbler—particles and a solid polymer material are tumbled together to coat the particles. A fluidized bed coater can also be used to coat the particles. In addition, the particles can simply be dipped into liquid or powdered polymer precursor. All of these techniques will be familiar to those skilled in the art.

In some embodiments, it is desirable to infiltrate a polymer or polymer precursor into the vascular and/or interstitial structure of bone-derived particles or into the bone-derived tissue itself. The vascular structure of bone includes such structures such as osteocyte lacunae, Haversian canals, Volksmann's canals, canaliculi and similar structures. The interstitial structure of the bone particles includes the spaces between trabeculae and similar features. Many of the monomers and other polymer precursors suggested for use with the invention are sufficiently flowable to penetrate through the channels and pores of trabecular bone. Some can even penetrate into the trabeculae or into the mineralized fibrils of cortical bone. Thus, it may only be necessary to incubate the bone particles in neat monomer or other polymer precursor for a period of time to accomplish infiltration. In certain embodiments, the polymer itself is sufficiently flowable that it can penetrate the channels and pores of bone. The polymer can also be heated or combined with a solvent to make it more flowable for this purpose. Other ceramic materials or bone-substitute materials employed as a particulate phase can also include porosity that can be infiltrated as described herein.

Vacuum infiltration can be used to help a polymer or precursor infiltrate the lacunae and canals, and, if desired, the canaliculi. Penetration of the microstructural channels of the bone particles will maximize the surface area of the interface between the particles and the polymer and prevent solvents and air bubbles from being trapped in the composite, e.g., between trabeculae. Vacuum infiltration, where appropriate, will also help remove air bubbles from the composite used to make the inventive bone anchors.

In another embodiment, infiltration is achieved using solvent infiltration. Vacuum infiltration may be inappropriate for highly volatile monomers. Solvents employed for infiltration should carefully selected, as many of the most common solvents used for infiltration are toxic. Highly volatile solvents such as acetone will evaporate during infiltration, reducing the risk that they will be incorporated into the polymer and implanted into the subject. Exemplary solvents for use in preparing the composite include but are not limited to dimethylsulfoxide (DMSO) and ethanol. As is well known to those skilled in the art, solvent infiltration is achieved by mixing the particles with solutions of the solvent with the polymer or polymer precursor, starting with very dilute solutions and proceeding to more concentrated solutions and finally to neat polymer or polymer precursor. Solvent infiltration can also provide improved tissue infiltration. In some embodiments, solvent infiltration is combined with pressure in vacuum; instead of finishing the infiltration with heat monomer, the pressure or vacuum is used to draw out the remaining solvent while pushing the polymer or polymer precursor even deeper into the particles.

One skilled in the art will recognize that other standard histological techniques, including pressure and heat, can be used to increase the infiltration of a polymer or polymer precursor into the particles. Infiltrated particles can then be combined with a volume of fresh polymer before administration. Automated apparatus for vacuum and pressure infiltration include the Tissue Tek VIP Vacuum infiltration processor E150/E300, available from Sakura Finetek, Inc.

Alternatively or in addition, a polymer or polymer precursor and particles can be supplied separately, e.g., in a kit, and mixed immediately prior to implantation or forming or molding an anchor. The kit can contain a preset supply of bone-derived and optionally other particles having, e.g., certain sizes, shapes, and levels of demineralization. The surface of the particles may have been optionally modified using one or more of the techniques described herein. Alternatively, the kit can provide several different types of particles of varying sizes, shapes, and levels of demineralization and that may have been chemically modified in different ways. A surgeon or other health care professional can also combine the components in the kit with autologous tissue derived during surgery or biopsy. For example, the surgeon may want to include autogenous tissue or cells, e.g., bone marrow or bone shavings generated while preparing the implant site, into the composite. The kit can further include one or more molds in the shape of the inventive anchors, and a surgeon can form the anchor in situ by pressing or injecting the composite into the mold. A mold shape, style or size can be selected based upon its suitability for the implant site.

The composite used to form the inventive anchors can include practically any ratio of polymer component and particles, for example, between about 5% and about 95% by weight of particles. For example, the composite can include about 50% to about 70% by weight particles. The desired proportion may depend on factors such as the placement site, the shape and size of the particles, how evenly the polymer is distributed among the particles, desired flowability of the composite, desired handling of the composite, desired moldability of the composite, and the mechanical and degradation properties of the composite. The proportions of the polymer and particles can influence various characteristics of the composite, for example, its mechanical properties, including fatigue strength, the degradation rate, and the rate of biological incorporation. In addition, the cellular response to the implanted anchor will vary with the proportion of polymer and particles. In some embodiments, the desired proportion of particles is determined not only by the desired biological properties of the implant but by the desired mechanical properties of the implant. That is, an increased proportion of particles will increase the viscosity of the composite, making it more difficult to mold or inject. A larger proportion of particles having a wide size distribution can give similar properties to a mixture having a smaller proportion of more evenly sized particles.

One skilled in the art will recognize that standard experimental techniques can be used to test biological and mechanical properties for a range of compositions. Such tests can enable optimization of a composite for a bone anchor useful in spinal surgery. For example, standard mechanical testing instruments can be used to test the compressive strength and stiffness of a candidate composite. Cells can be cultured on the composite for an appropriate period of time and the metabolic products and the amount of proliferation (e.g., the number of cells in comparison to the number of cells seeded) analyzed. The weight change of the candidate composite can be measured after incubation in saline or other fluids. Repeated analysis will demonstrate whether degradation of the composite is linear or not, and mechanical testing of the incubated material will show the change in mechanical properties as the candidate composite degrades. Such testing can also be used to compare the enzymatic and non-enzymatic degradation of the composite and to determine the levels of enzymatic degradation. A composite that is degraded is transformed into living bone upon implantation. A non-degradable composite leaves a supporting scaffold which can be interpenetrated with bone or other tissue. A complete evaluation of test results can enable the selection of a particular composite for making an inventive anchor suitable for a particular implant site.

### Selection of Polymer

Any biocompatible polymer can be used in preparing the composites of the invention. Biodegradable polymers may be preferable in some embodiments because composite bone anchors made from such materials can be transformed into living bone. Polymers that do not degrade may be preferred for some applications, as they leave a supporting scaffold through which new living tissue can interpenetrate. Co-polymers and/or polymer blends can also be used in preparing the composites for the inventive bone anchors. The selected polymer can be formable and settable under particular conditions, or a monomer or pre-polymer of the polymer can be used. In certain embodiments, the composite becomes more formable when heated to or over a particular temperature, for example, a temperature at or above the glass transition temperature or melting point of the polymer component. Alternatively, the composite can be more formable when the polymer component has a certain cross-link density. After the composite is molded or injected, the cross-link density of the polymer component of the composite can be increased to solidify or set the composite. In another embodiment, a small amount of monomer is mixed with the polymeric and bone components of the composite. Upon exposure to an energy source, e.g., UV light or heat, the monomer and polymer will further polymerize and/or cross-link, increasing the molecular weight, the cross-link density, or both. Alternatively or in addition, exposure to body heat, a chemical agent, or physiological fluids can stimulate polymerization.

If heat is employed to render the composite and/or the polymer component of the composite formable, the glass transition T_{g} or melting temperature of the polymer component is preferably higher than normal body temperature, for example, higher than about 40° C. Polymers that become more formable at higher temperatures, e.g., higher than about 45° C., higher than about 50° C., higher than about 55° C., higher than about 60° C., higher than about 70° C., higher than about 80° C., higher than about 90° C., higher than about 100° C., higher than about 110° C., or higher than about 120° C. can also be used. However, the temperature required for rendering the composite formable should not be so high as to cause unacceptable tissue necrosis upon implantation. Prior to implantation, the composite is typically sufficiently cooled to cause little or no tissue necrosis upon implantation. Exemplary polymers having T_{g} suitable for use with the invention include but are not limited to starch-poly(caprolactone), poly(caprolactone), poly(lactide), poly(D,L-lactide), poly(lactide-co-glycolide), poly(D,L-lactide-co-glycolide), polycarbonates, polyurethane, tyrosine polycarbonate, tyrosine polyarylate, poly(orthoesters), polyphosphazenes, polypropylene fumarate, polyhydroxyvalerate, polyhydroxy butyrate, acrylates, methacrylates, and co-polymers, mixtures, enantiomers, and derivatives thereof. In certain particular embodiments, the polymer is starch-poly(caprolactone), poly(caprolactone), poly(lactide), poly(D,L-lactide), poly(lactide-co-glycolide), poly(D,L-lactide-co-glycolide), polyurethane, or a co-polymer, mixture, enantiomer, stereoisomer, or derivative thereof. In certain embodiments, the polymer is poly(D,L-lactide). In certain other embodiments, the polymer is poly(D,L-lactide-co-glycolide). In certain embodiments, the polymer is poly(caprolactone). In certain embodiments, the polymer is a poly(urethane). In certain embodiments, the polymer is tyrosine polycarbonate. In certain embodiments, the polymer is tyrosine polyarylate.

It is not necessary for all such embodiments that the glass transition temperature of the polymer be higher than body temperature. In non-load bearing and some load-bearing applications, the viscosity of the polymer component and resulting composite need only be high enough at body temperature that the composite will not flow out of the implant site. In other embodiments, the polymer component may have crystalline and non-crystalline regions. Depending on the ratio of crystalline and non-crystalline material, a polymer may remain relatively rigid between the glass transition and melting temperatures. Indeed, for some polymers, the melting temperature will determine when the polymer material becomes formable.

Since the composite can be rendered formable prior to implantation of the inventive anchors, polymer components with glass transition or melting temperatures higher than 80° C. are also suitable for use with the invention, despite the sensitivity of biological material to heat. For example, PMMA bone cement achieves temperatures of about 50-60° C. during curing. Potential damage to bone and/or other materials in the composite depends on both the temperature and the processing time. As the T_{g} or Tₘ of the polymer component increases, the composite should be heated for shorter periods of time to minimize damage to its biological components and should cool sufficiently quickly to minimize injury at the implantation site.

The T_{g} of a polymer can be manipulated by adjusting its cross-link density and/or its molecular weight. Thus, for polymers whose glass transition temperatures are not sufficiently high, increasing the cross-link density or molecular weight can increase the T_{g} to a level at which composites containing these polymers can be heated to render them formable. Alternatively, the polymer can be produced with crystalline domains, increasing the stiffness of the polymer at temperatures above its glass transition temperature. In addition, the T_{g} of the polymer component can be modified by adjusting the percentage of the crystalline component. Increasing the volume fraction of the crystalline domains can so reduce the formability of the polymer between T_{g} and Tₘ that the composite has to be heated above its melting point to be sufficiently formable for use in accordance with the present invention.

Where a monomer, prepolymer, or other partially polymerized or partially cross-linked polymer is employed in preparing the composite, the resulting polymer can form by step or chain polymerization. One skilled in the art will recognize that the rate of polymerization should be controlled so that any change in volume upon polymerization does not impact mechanical stresses on the included bone particles. The amount and kind of radical initiator, e.g., photo-active initiator (e.g., UV, infrared, or visible), thermally-active initiator, or chemical initiator, or the amount of heat or light employed, can be used to control the rate of reaction or modify the molecular weight. Where desired, a catalyst can be used to increase the rate of reaction or modify the molecular weight. For example, a strong acid can be used as a catalyst for step polymerization. Exemplary catalysts for ring opening polymerization include organotin compounds and glycols and other primary alcohols. Trifunctional and other multifunctional monomers or cross-linking agents can also be used to increase the cross-link density. For chain polymerizations, the concentration of a chemical initiator in the monomer-bone particle mixture can be adjusted to manipulate the final molecular weight.

Exemplary initiators are listed in George Odian's Principles of Polymerization, (3rd Edition, 1991, New York, John Wiley and Sons) and available from companies such as Polysciences, Wako Specialty Chemicals, Akzo Nobel, and Sigma. Polymerization initiators useful in the composite include organic peroxides (e.g., benzoyl peroxide) and azo initiators (e.g., AIBN). Preferably, the initiator like the polymer and/or monomer is biocompatible. Alternatively, polymerized or partially polymerized material can be exposed to UV light, microwaves, or an electron beam to provide energy for inter-chain reactions. Polymerization can also be triggered by exposure to physiological temperatures or fluids. One skilled in the art will recognize how to modify the cross-link density to control the rate of degradation and the stiffness of the inventive bone anchor. For example, an accelerator such as an N,N-dialkyl aniline or an N,N-dialkyl toluidine can be used. Exemplary methods for controlling the rate of polymerization and the molecular weight of the product are also described in Odian (1991), the entire contents of which are incorporated herein by reference.

Any biocompatible polymer can be used to form composites for use in accordance with the present invention. As noted above, the cross-link density and molecular weight of the polymer may need to be manipulated so that the polymer can be formed and set when desired. In some embodiments, the formable polymer material includes monomers, low-molecular weight chains, oligomers, or telechelic chains of the polymers described herein, and these are cross-linked or further polymerized after implantation. A number of biodegradable and non-biodegradable biocompatible polymers are known in the field of polymeric biomaterials, controlled drug release, and tissue engineering (see, for example, U.S. Pat. Nos. 6,123,727; 5,804,178; 5,770,417; 5,736,372; 5,716,404 to Vacanti; 6,095,148; 5,837,752 to Shastri; 5,902,599 to Anseth; 5,696,175; 5,514,378; 5,512,600 to Mikos; 5,399,665 to Barrera; 5,019,379 to Domb; 5,010,167 to Ron; 4,946,929 to d'Amore; and 4,806,621; 4,638,045 to Kohn; Beckamn et al., U.S. Patent Application 2005/0013793, published Jan. 20, 2005; see also Langer, Acc. Chem. Res. 33:94, 2000; Langer, J. Control Release 62:7, 1999; and Uhrich et al., Chem. Rev. 99:3181, 1999, the contents of all of which are incorporated herein by reference).

Other polymers useful in preparing composites in accordance with the present invention are described in U.S. Pat. No. 7,291,345, issued Nov. 6, 2007, entitled “Formable and settable polymer bone composite and method of production thereof;” U.S. Pat. No. 7,270,813 issued Sep. 18, 2007, entitled “Coupling agents for orthopedic biomaterials;” and U.S. Ser. No. 60/760,538, filed on Jan. 19, 2006 and entitled “Injectable and Settable Bone Substitute Material,” also filed as international application PCT/US07/01540, filed Jan. 19, 2007 all of which are incorporated herein by reference.

In one embodiment, the polymer used in the composite is biodegradable. Exemplary biodegradable materials include lactide-glycolide copolymers of any ratio (e.g., 85:15, 40:60, 30:70, 25:75, or 20:80), poly(L-lactide-co-D,L-lactide), polyglyconate, poly(arylates), poly(anhydrides), poly(hydroxy acids), polyesters, poly(ortho esters), poly(alkylene oxides), polycarbonates, poly(propylene fumarates), poly(propylene glycol-co fumaric acid), poly(caprolactones), polyamides, polyesters, polyethers, polyureas, polyamines, polyamino acids, polyacetals, poly(orthoesters), poly(pyrolic acid), poly(glaxanone), poly(phosphazenes), poly(organophosphazene), polylactides, polyglycolides, poly(dioxanones), polyhydroxybutyrate, polyhydroxyvalyrate, polyhydroxybutyrate/valerate copolymers, poly(vinyl pyrrolidone), biodegradable polycyanoacrylates, biodegradable polyurethanes including glucose-based polyurethanes and lysine-based polyurethanes, and polysaccharides (e.g., chitin, starches, celluloses). In certain embodiments, the polymer used in the composite is poly(lactide-co-glycolide). The ratio of lactide and glycolide units in the polymer can vary. Particularly useful ratios are approximately 45%-80% lactide to approximately 44%-20% glycolide. In certain embodiments, the ratio is approximately 50% lactide to approximately 50% glycolide. In other certain embodiments, the ratio is approximately 65% lactide to approximately 45% glycolide. In other certain embodiments, the ratio is approximately 60% lactide to approximately 40% glycolide. In other certain embodiments, the ratio is approximately 70% lactide to approximately 30% glycolide. In other certain embodiments, the ratio is approximately 75% lactide to approximately 25% glycolide. In certain embodiments, the ratio is approximately 80% lactide to approximately 20% glycolide. In certain of the above embodiments, lactide is D,L-lactide. In other embodiments, lactide is L-lactide. In certain particular embodiments, RESOMER® 824 (poly-L-lactide-co-glycolide) (Boehringer Ingelheim) is incorporated as the polymer in the composite used to make the inventive bone anchors. In certain particular embodiments, RESOMER® 504 (poly-D,L-lactide-co-glycolide) (Boehringer Ingelheim) is used as the polymer in the composite. In certain particular embodiments, PURASORB PLG (75/25 poly-L-lactide-co-glycolide) (Purac Biochem) is used as the polymer in the composite. In certain particular embodiments, PURASORB PG (polyglycolide) (Purac Biochem) is used as the polymer in the composite. In certain embodiments, the polymer is PEGylated-poly(lactide-co-glycolide). In certain embodiments, the polymer is PEGylated-poly(lactide). In certain embodiments, the polymer is PEGylated-poly(glycolide). In other embodiments, the polymer is polyurethane. In other embodiments, the polymer is polycaprolactone. In certain embodiments, the polymer is a co-polymer of poly(caprolactone) and poly(lactide).

For polyesters such as poly(lactide) and poly(lactide-co-glycolide), the inherent viscosity of the polymer ranges from about 0.4 dL/g to about 5 dL/g. In certain embodiments, the inherent viscosity of the polymer ranges from about 0.6 dL/g to about 2 dL/g. In certain embodiments, the inherent viscosity of the polymer ranges from about 0.6 dL/g to about 3 dL/g. In certain embodiments, the inherent viscosity of the polymer ranges from about 1 dL/g to about 3 dL/g. In certain embodiments, the inherent viscosity of the polymer ranges from about 0.4 dL/g to about 1 dL/g. For poly(caprolactone), the inherent viscosity of the polymer ranges from about 0.5 dL/g to about 1.5 dL/g. In certain embodiments, the inherent viscosity of the poly(caprolactone) ranges from about 1.0 dL/g to about 1.5 dL/g. In certain embodiments, the inherent viscosity of the poly(caprolactone) ranges from about 1.0 dL/g to about 1.2 dL/g. In certain embodiments, the inherent viscosity of the poly(caprolactone) is about 1.08 dL/g.

Natural polymers, including collagen, polysaccharides, agarose, glycosaminoglycans, alginate, chitin, and chitosan, can also be employed in preparing the composite. Tyrosine-based polymers, including but not limited to polyarylates and polycarbonates, can also be employed (see Pulapura, et al., “Tyrosine-derived polycarbonates: Backbone-modified “pseudo”-poly(amino acids) designed for biomedical applications,” Biopolymers, 1992, 32: 411-417; Hooper, et al., “Diphenolic monomers derived from the natural amino acid α-L-tyrosine: an evaluation of peptide coupling techniques,” J. Bioactive and Compatible Polymers, 1995, 10:327-340, the contents of both of which are incorporated herein by reference). Monomers for tyrosine-based polymers can be prepared by reacting an L-tyrosine-derived diphenol compound with phosgene or a diacid (Hooper, 1995; Pulapura, 1992). Similar techniques can be used to prepare amino acid-based monomers of other amino acids having reactive side chains, including imines, amines, thiols, etc. The polymers described in U.S. patent application Ser. No. 11/336,127, filed Jan. 19, 2006 can also be used in embodiments of the present invention. In one embodiment, the degradation products include bioactive materials, biomolecules, small molecules, or other such materials that participate in metabolic processes.

Polymers can be manipulated to adjust their degradation rates. The degradation rates of polymers are well characterized in the literature (see Handbook of Biodegradable Polymers, Domb, et al., eds., Harwood Academic Publishers, 1997, the entire contents of which are incorporated herein by reference). In addition, increasing the cross-link density of a polymer tends to decrease its degradation rate. The cross-link density of a polymer can be manipulated during polymerization by adding a cross-linking agent or promoter. After polymerization, cross-linking can be increased by exposure to UV light or other radiation. Co-monomers or mixtures of polymers, for example, lactide and glycolide polymers, can be employed to manipulate both degradation rate and mechanical properties of the inventive anchors.

Non-biodegradable polymers can also be incorporated in the composite used to make the inventive bone anchors. For example, polypyrrole, polyanilines, polythiophene, and derivatives thereof are useful electroactive polymers that can transmit voltage from endogenous bone to an implant. Other non-biodegradable, yet biocompatible polymers include polystyrene, non-biodegradable polyesters, non-biodegradable polyureas, poly(vinyl alcohol), non-biodegradable polyamides, poly(tetrafluoroethylene), and expanded polytetrafluoroethylene (ePTFE), poly(ethylene vinyl acetate), polypropylene, non-biodegradable polyacrylate, non-biodegradable polycyanoacrylates, non-biodegradable polyurethanes, mixtures and copolymers of poly(ethyl methacrylate) with tetrahydrofurfuryl methacrylate, polymethacrylate, non-biodegradable poly(methyl methacrylate), polyethylene (including ultra high molecular weight polyethylene (UHMWPE)), polypyrrole, polyanilines, polythiophene, poly(ethylene oxide), poly(ethylene oxide co-butylene terephthalate), poly ether-ether ketones (PEEK), and polyetherketoneketones (PEKK). Monomers that are used to produce any of these polymers are easily purchased from companies such as Polysciences, Sigma, and Scientific Polymer Products.

Those skilled in the art will recognize that this is an exemplary, not a comprehensive, list of polymers appropriate for in vivo applications. Co-polymers, mixtures, and adducts of the above polymers can also be used with the invention.

### Non-Composite Anchors

In certain embodiments, inventive bone anchors can be formed from substantially a single type of a wide variety of biocompatible materials. The material can be non-resorbable, non-biodegradable, resorbable, or biodegradable. The material can be polymeric, ceramic, glass, or metal. In some embodiments, the inventive bone anchors are made from a material comprising calcium phosphate, silicate-substituted calcium phosphate, calcium sulfate, Bioglass, etc. The material can be organic or inorganic.

Non-biodegradable polymers can include, polypyrrole, polyanilines, polythiophene, and derivatives thereof are useful electroactive polymers that can transmit voltage from endogenous bone to an implant. Other non-biodegradable, yet biocompatible polymers include polystyrene, polyesters, polyureas, poly(vinyl alcohol), polyamides, poly(tetrafluoroethylene), and expanded polytetrafluoroethylene (ePTFE), poly(ethylene vinyl acetate), polypropylene, polyacrylate, non-biodegradable polycyanoacrylates, non-biodegradable polyurethanes, mixtures and copolymers of poly(ethyl methacrylate) with tetrahydrofurfuryl methacrylate, polymethacrylate, poly(methyl methacrylate), polyethylene (including ultra high molecular weight polyethylene (UHMWPE)), polypyrrole, polyanilines, polythiophene, poly(ethylene oxide), poly(ethylene oxide co-butylene terephthalate), poly ether-ether ketones (PEEK), and polyetherketoneketones (PEKK). Monomers that are used to produce any of these polymers are easily purchased from companies such as Polysciences, Sigma, and Scientific Polymer Products. In some embodiments, an inventive bone anchor is formed from bone cement, e.g., a material comprised primarily of poly(methylmethacrylate) (PMMA).

Exemplary biodegradable materials include lactide-glycolide copolymers of any ratio (e.g., 85:15, 40:60, 30:70, 25:75, or 20:80), poly(L-lactide-co-D,L-lactide), polyglyconate, poly(arylates), poly(anhydrides), poly(hydroxy acids), polyesters, poly(ortho esters), poly(alkylene oxides), polycarbonates, poly(propylene fumarates), poly(propylene glycol-co fumaric acid), poly(caprolactones), polyamides, polyesters, polyethers, polyureas, polyamines, polyamino acids, polyacetals, poly(orthoesters), poly(pyrolic acid), poly(glaxanone), poly(phosphazenes), poly(organophosphazene), polylactides, polyglycolides, poly(dioxanones), polyhydroxybutyrate, polyhydroxyvalyrate, polyhydroxybutyrate/valerate copolymers, poly(vinyl pyrrolidone), biodegradable polycyanoacrylates, biodegradable polyurethanes including glucose-based polyurethanes and lysine-based polyurethanes, and polysaccharides (e.g., chitin, starches, celluloses). In certain embodiments, the polymer used in the composite is poly(lactide-co-glycolide). The ratio of lactide and glycolide units in the polymer can be varied selectively. Particularly useful ratios are approximately 45%-80% lactide to approximately 44%-20% glycolide. In certain embodiments, the ratio is approximately 50% lactide to approximately 50% glycolide. In other certain embodiments, the ratio is approximately 65% lactide to approximately 45% glycolide. In other certain embodiments, the ratio is approximately 60% lactide to approximately 40% glycolide. In other certain embodiments, the ratio is approximately 70% lactide to approximately 30% glycolide. In other certain embodiments, the ratio is approximately 75% lactide to approximately 25% glycolide. In certain embodiments, the ratio is approximately 80% lactide to approximately 20% glycolide. In certain of the above embodiments, lactide is D,L-lactide. In other embodiments, lactide is L-lactide. In certain particular embodiments, RESOMER® 824 (poly-L-lactide-co-glycolide) (Boehringer Ingelheim) is incorporated as the polymer in the composite used to make the inventive bone anchors. In certain particular embodiments, RESOMER® 504 (poly-D,L-lactide-co-glycolide) (Boehringer Ingelheim) is used as the polymer in the composite. In certain particular embodiments, PURASORB PLG (75/25 poly-L-lactide-co-glycolide) (Purac Biochem) is used as the polymer in the composite. In certain particular embodiments, PURASORB PG (polyglycolide) (Purac Biochem) is used as the polymer in the composite. In certain embodiments, the polymer is PEGylated-poly(lactide-co-glycolide). In certain embodiments, the polymer is PEGylated-poly(lactide). In certain embodiments, the polymer is PEGylated-poly(glycolide). In other embodiments, the polymer is polyurethane. In other embodiments, the polymer is polycaprolactone. In certain embodiments, the polymer is a co-polymer of poly(caprolactone) and poly(lactide). For polyesters such as poly(lactide) and poly(lactide-co-glycolide), the inherent viscosity of the polymer ranges from about 0.4 dL/g to about 5 dL/g. In certain embodiments, the inherent viscosity of the polymer ranges from about 0.6 dL/g to about 2 dL/g. In certain embodiments, the inherent viscosity of the polymer ranges from about 0.6 dL/g to about 3 dL/g. In certain embodiments, the inherent viscosity of the polymer ranges from about 1 dL/g to about 3 dL/g. In certain embodiments, the inherent viscosity of the polymer ranges from about 0.4 dL/g to about 1 dL/g. For poly(caprolactone), the inherent viscosity of the polymer ranges from about 0.5 dL/g to about 1.5 dL/g. In certain embodiments, the inherent viscosity of the poly(caprolactone) ranges from about 1.0 dL/g to about 1.5 dL/g. In certain embodiments, the inherent viscosity of the poly(caprolactone) ranges from about 1.0 dL/g to about 1.2 dL/g. In certain embodiments, the inherent viscosity of the poly(caprolactone) is about 1.08 dL/g. Natural polymers, including collagen, polysaccharides, agarose, glycosaminoglycans, alginate, chitin, and chitosan, can also be employed in preparing the composite. Tyrosine-based polymers, including but not limited to polyarylates and polycarbonates, can also be employed (see Pulapura, et al., “Tyrosine-derived polycarbonates: Backbone-modified “pseudo”-poly(amino acids) designed for biomedical applications,” Biopolymers, 1992, 32: 411-417; Hooper, et al., “Diphenolic monomers derived from the natural amino acid α-L-tyrosine: an evaluation of peptide coupling techniques,” J. Bioactive and Compatible Polymers, 1995, 10:327-340, the contents of both of which are incorporated herein by reference). Monomers for tyrosine-based polymers can be prepared by reacting an L-tyrosine-derived diphenol compound with phosgene or a diacid (Hooper, 1995; Pulapura, 1992). Similar techniques can be used to prepare amino acid-based monomers of other amino acids having reactive side chains, including imines, amines, thiols, etc. The polymers described in U.S. patent application Ser. No. 11/336,127, filed Jan. 19, 2006 and published as 2006/0216323 can also be used in embodiments of the present invention. In one embodiment, the degradation products include bioactive materials, biomolecules, small molecules, or other such materials that participate in metabolic processes.

Examples of biocompatible ceramics include porcelain, alumina, hydroxyapatite, calcium pyrophosphate, tricalcium phosphate, calcium carbonate, and zirconia. Ceramics can be formed into a bone anchor by machining methods. Examples of biocompatible metals include gold, silver, titanium, titanium alloys, cobalt chrome alloys, aluminum, aluminum alloys, stainless steel, and stainless steel alloys. Metals can be formed into the inventive bone-anchor shapes by machining or casting.

### Additional Components

Additional materials can be included in the composite or non-composite bone anchors used to prepare the inventive bone anchors. The additional material can be biologically active or inert. Additional materials can also be added to the composite or non-composite anchors to improve their chemical, mechanical, or biophysical properties. Additional materials can also be added to improve the handling or storage of the composite or non-composite anchors (e.g., a preservative, a sterilizing agent). Those of skill in this art will appreciate the myriad of different components that may be included in the composite or non-composite bone anchors.

Additional components or additives can be any type of chemical compound including proteins, peptides, polynucleotides (e.g., vectors, plasmids, cosmids, artificial chromosomes, etc.), lipids, carbohydrates, organic molecules, small molecules, organometallic compounds, metals, ceramics, polymers, etc. Living cells, tissue samples, or viruses can also be added to the composites. In certain embodiments, the additional material comprises cells, which may optionally be genetically engineered. For example, the cells can be engineered to produce a specific growth factor, chemotactic factor, osteogenic factor, etc. In certain embodiments, the cells are engineered to produce a polynucleotide such as an siRNA, shRNA, RNAi, microRNA, etc. The cell can include a plasmid, or other extra-chromosomal piece of DNA. In certain embodiments, a recombinant construct is integrated into the genome of the cell. In certain embodiments, the additional material comprises a virus. Again, the virus can be genetically engineered. Tissues such as bone marrow and bone samples can be combined with a composite of polymer and bone-derived particles, or a non-composite of polymer, ceramic or metal. The composite or non-composite can include additional calcium-based ceramics such as calcium phosphate and calcium carbonate. In certain embodiments, non-biologically active materials are incorporated into the composite or non-composite. For example, labeling agents such as radiopaque, luminescent, or magnetically active particles can be attached to the bone-derived particles using silane chemistry or other coupling agents, for example zirconates and titanates, or mixed into the polymer, as described herein. Alternatively, or in addition, poly(ethylene glycol) (PEG) can be attached to the bone particles. Biologically active molecules, for example, small molecules, bioactive agents, and biomolecules such as lipids can be linked to the particles through silane SAMs or using a polysialic acid linker (see, for example, U.S. Pat. No. 5,846,951; incorporated herein by reference). In some embodiments, labeling agents and biologically active molecules are added to non-composite materials.

The composite or non-composite used for preparing the bone anchors can also include one or more other components such as a plasticizer. Plasticizer are typically compounds added to polymers or plastics to soften them or make them more pliable. Plasticizers soften, make workable, or otherwise improve the handling properties of a polymer or composite. In certain embodiments, plasticizers allow the composite or non-composite anchors to be moldable at a lower temperature, thereby avoiding heat induced tissue necrosis during implantation. The plasticizer can evaporate or otherwise diffuse out of the composite over time, thereby allowing the composite or non-composite anchor to harden or set. Plasticizers are thought to work by embedding themselves between the chains of polymers. This forces the polymer chains apart and thus lowers the glass transition temperature of the polymer. Typically, the more plasticizer that is added, the more flexible the resulting composite or non-composite polymer will be.

In certain embodiments, the plasticizer is based on an ester of a polycarboxylic acid with linear or branched aliphatic alcohols of moderate chain length. For example, some plasticizers are adipate-based. Examples of adipate-based pasticizers include bis(2-ethylhexyl)adipate (DOA), dimethyl adipate (DMAD), monomethyl adipate (MMAD), and dioctyl adipate (DOA). Other plasticizers are based on maleates, sebacates, or citrates such as bibutyl maleate (DBM), diisobutylmaleate (DIBM), dibutyl sebacate (DBS), triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC), and trimethylcitrate (TMC). Other plasticizers are phthalate based. Examples of phthalate-based plasticizers are N-methyl phthalate, bis(2-ethylhexyl)phthalate (DEHP), diisononyl phthalate (DINP), bis(n-butyl)phthalate (DBP), butyl benzyl phthalate (BBzP), diisodecyl phthalate (DOP), diethyl phthalate (DEP), diisobutyl phthalate (DIBP), and di-n-hexyl phthalate. Other suitable plasticizers include liquid polyhydroxy compounds such as glycerol, polyethylene glycol (PEG), triethylene glycol, sorbitol, monacetin, diacetin, and mixtures thereof. Other plasticizers include trimellitates (e.g., trimethyl trimellitate (TMTM), tri-(2-ethylhexyl) trimellitate (TEHTM-MG), tri-(n-octyl,n-decyl) trimellitate (ATM), tri-(heptyl,nonyl) trimellitate (LTM), n-octyl trimellitate (OTM)), benzoates, epoxidized vegetable oils, sulfonamides (e.g., N-ethyl toluene sulfonamide (ETSA), N-(2-hydroxypropyl)benzene sulfonamide (HP BSA), N-(n-butyl) butyl sulfonamide (BBSA-NBBS)), organophosphates (e.g., tricresyl phosphate (TCP), tributyl phosphate (TBP)), glycols/polyethers (e.g., triethylene glycol dihexanoate, tetraethylene glycol diheptanoate), and polymeric plasticizers. Other plasticizers are described in Handbook of Plasticizers (G. Wypych, Ed., ChemTec Publishing, 2004), which is incorporated herein by reference. In certain embodiments, other polymers are added to the composite or non-composite as plasticizers. In certain particular embodiments, polymers with the same chemical structure as those used in the composite or non-composite are used but with lower molecular weights to soften the overall composite or non-composite. In certain embodiments, oligomers or monomers of the polymers used in the composite or non-composite are used as plasticizers. In other embodiments, different polymers with lower melting points and/or lower viscosities than those of the polymer component of the composite or non-composite are used. In certain embodiments, oligomers or monomers of polymers different from those used in the composite or non-composite are used as plasticizers. In certain embodiments, the polymer used as a plasticizer is poly(ethylene glycol) (PEG). The PEG used as a plasticizer is typically a low molecular weight PEG such as those having an average molecular weight of 1000 to 10000 g/mol, preferably from 4000 to 8000 g/mol. In certain embodiments, PEG 4000 is used in the composite or non-composite. In certain embodiments, PEG 5000 is used in the composite or non-composite. In certain embodiments, PEG 6000 is used in the composite or non-composite. In certain embodiments, PEG 7000 is used in the composite or non-composite. In certain embodiments, PEG 8000 is used in the composite or non-composite. The plasticizer (PEG) is particularly useful in making more moldable composites or non-composite polymers that include poly(lactide), poly(D,L-lactide), poly(lactide-co-glycolide), poly(D,L-lactide-co-glycolide), or poly(caprolactone). In certain embodiments, PEG is grafted onto a polymer of the composite or non-composite polymer or is co-polymerized with the polymers of the composite or non-composite.

Plasticizer can comprise 1%-40% by weight of the composite or non-composite used to make the inventive bone anchors. In certain embodiments, the plasticizer is 10%-30% by weight. In certain embodiments, the plasticizer is approximately 10% by weight. In certain embodiments, the plasticizer is approximately 15% by weight. In certain embodiments, the plasticizer is approximately 20% by weight. In certain embodiments, the plasticizer is approximately 25% by weight. In certain embodiments, the plasticizer is approximately 30% by weight. In certain embodiments, the plasticizer is approximately 33% by weight. In certain embodiments, the plasticizer is approximately 40% by weight. In certain embodiments, a plasticizer is not used in the composite or non-composite. For example, in some polycaprolactone-containing composites or non-composite polymers, a plasticizer is not used.

In some embodiments, polymers or materials that expand upon absorbing water are incorporated into the composite or non-composite polymer used to make the bone anchors. Any of the above-mentioned polymers which expand upon absorption of water can be used for these embodiments. For such composites, hygroscopic expansion of the bone anchor can push portions of the anchor into better contact with the surrounding bone improving its anchoring at the implant site.

The inventive composite or non-composite bone anchor can be porous (e.g., at the time of manufacture), can be made porous prior to implantation, can incorporate porous materials, or can become porous upon implantation. For a general discussion of the use of porosity in osteoimplants, see U.S. patent application US 2005/0251267, published Nov. 10, 2005; which is incorporated herein by reference. A porous implant with an interconnecting network of pores has been shown to facilitate the invasion of cells and promote the organized growth of incoming cells and tissue (e.g., living bone). Allcock et al. “Synthesis of poly[(amino acid alkyl ester) phosphazenes” Macromolecules 10:824-830, 1977; Allcock et al. “Hydrolysis pathways for aminophosphazenes” Inorg. Chem. 21:515-521, 1982; Mikos et al. “Prevascularization of biodegradable polymer scaffolds for hepatocyte transplantation” Proc. ACS Div. of Polymer Mater. 66:33, 1992; Eggli et al. “Porous hydroxyapatite and tricalcium phosphate cylinders with two different pore size ranges implanted in the cancellous bone of rabbits” Clin. Orthop. 232:127-138, 1987; each of which is incorporated herein by reference. Porosity has also been shown to influence the biocompatibility and bony integration of polymeric composites. White et al. “Biomaterial aspects of Interpore 200 porous hydroxyapatite” Dental Clinics of N. Amer. 30:49-67, 1986; which is incorporated herein by reference.

A porous bone anchor can include either or both open and closed cells. The terms “open cells” and “open-celled structure” are used herein interchangeably and refer to a porous material with very large permeability, and where no significant surface barriers exist between cells (i.e., where the pores are connected). The terms “closed cells” and “close-celled structure” are used herein interchangeably and refer to a porous material where the pores are not connected, resulting in a weakly permeable material. Open cells in a bone anchor increase the paths for tissue to infiltrate the composite or non-composite material and will decrease degradation times. The proportion and size distribution ranges of open and closed cells within the inventive bone anchors (e.g., before or after implantation) can be adjusted by controlling such factors as the identity of the porogen, percentage of porogen, percentage of particles, the properties of the polymer, etc.

The bone anchors of the present invention can exhibit high degrees of porosity over a wide range of effective pore sizes. Thus, bone anchors of the present invention can have, at once, macroporosity, mesoporosity and microporosity. Macroporosity is characterized by pore diameters greater than about 100 microns. Mesoporosity is characterized by pore diameters between about 100 microns about 10 microns; and microporosity occurs when pores have diameters below about 10 microns. In some embodiments, the bone anchor has a porosity of at least about 30%. For example, in certain embodiments, the bone anchor has a porosity of more than about 50%, more than about 60%, more than about 70%, more than about 80%, or more than about 90%. When expressed in this manner, a porosity of N % means that N % of the volume of the bone anchor composite comprises porous vacancies, porous material, or porogens. Advantages of a highly porous bone anchor over less porous or non-porous anchor include, but are not limited to, more extensive cellular and tissue in-growth into the anchor, more continuous supply of nutrients, more thorough infiltration of therapeutics, and enhanced revascularization, allowing bone growth and repair to take place more efficiently. Furthermore, in certain embodiments, the porosity of the bone anchor is used to load the anchor with biologically active agents such as drugs, small molecules, cells, peptides, polynucleotides, growth factors, osteogenic factors, etc, for delivery at the implant site. Porosity can also render certain embodiments of the present invention compressible.

In certain particular embodiments, the pores of the composite or non-composite comprising the inventive bone anchors are preferably over 100 microns wide for the invasion of cells and bony in-growth. Klaitwatter et al. “Application of porous ceramics for the attachment of load bearing orthopedic applications” J. Biomed. Mater. Res. Symp. 2:161, 1971; each of which is incorporated herein by reference. In certain embodiments, the pore size ranges from approximately 50 microns to approximately 500 microns, preferably from approximately 100 microns to approximately 250 microns.

The porosity of the bone anchor can be accomplished using any means known in the art. Exemplary methods of creating porosity in a material used to make the bone anchor include, but are not limited to, particular leaching processes, gas foaming processing, supercritical carbon dioxide processing, sintering, phase transformation, freeze-drying, cross-linking, molding, porogen melting, polymerization, melt-blowing, and salt fusion (Murphy et al. Tissue Engineering 8(1):43-52, 2002; incorporated herein by reference). For a review, see Karageorgiou et al., Biomaterials 26:5474-5491, 2005; incorporated herein by reference. The porosity can be a feature of the material during manufacture or before implantation, or the porosity may only be available after implantation. For example, an implanted bone anchor can include latent pores. These latent pores can arise from including porogens in the composite.

The porogen can be any chemical compound that will reserve a space within the composite or non-composite material while being molded into a bone anchor and will diffuse, dissolve, and/or degrade prior to or after implantation of the anchor leaving a pore in the material. Porogens preferably have the property of not being appreciably changed in shape and/or size during the procedure to make the inventive bone anchor, or to make the anchor formable or moldable. For example, the porogen should retain its shape during the heating of the composite or non-composite to make it moldable. Therefore, the porogen preferably does not melt upon heating of the material to make it moldable. In certain embodiments, the porogen has a melting point greater than about 60° C., greater than about 70° C., greater than about 80° C., greater than about 85° C., or greater than about 90° C.

Porogens can be of any shape or size. The porogen can be spheroidal, cuboidal, rectangular, elonganted, tubular, fibrous, disc-shaped, platelet-shaped, polygonal, etc. In certain embodiments, the porogen is granular with a diameter ranging from approximately 100 microns to approximately 800 microns. In certain embodiments, the porogen is elongated, tubular, or fibrous. Such porogens provide increased connectivity of the pores within the composite or non-composite material and/or also allow for a lesser percentage of the porogen in the composite. The amount of the porogen can be varied selectively in the composite from 1% to 80% by weight. In certain embodiments, the porogen makes up from about 5% to about 80% by weight of the composite or non-composite material. In certain embodiments, the porogen makes up from about 10% to about 50% by weight of the material. Pores in the composite are thought to improve the osteoinductivity or osteoconductivity of the composite by providing holes for cells such as osteoblasts, osteoclasts, fibroblasts, cells of the osteoblast lineage, stem cells, etc. The pores provide the bone-anchor material with biological in-growth capacity. Pores in the composite or non-composite can also provide for easier degradation of the material as bone is formed and/or remodeled. Preferably, the porogen is biocompatible.

The porogen can be a gas, liquid, or solid. Exemplary gases that can act as porogens include carbon dioxide, nitrogen, argon, or air. Exemplary liquids include water, organic solvents, or biological fluids (e.g., blood, lymph, plasma). The gaseous or liquid porogen can diffuse out of the bone anchor before or after implantation thereby providing pores for biological in-growth. Solid porogens can be crystalline or amorphous. Examples of possible solid porogens include water soluble compounds. In certain embodiments, the water soluble compound has a solubility of greater than 10 g per 100 mL water at 25° C. In certain embodiments, the water soluble compound has a solubility of greater than 25 g per 100 mL water at 25° C. In certain embodiments, the water soluble compound has a solubility of greater than 50 g per 100 mL water at 25° C. In certain embodiments, the water soluble compound has a solubility of greater than 75 g per 100 mL water at 25° C. In certain embodiments, the water soluble compound has a solubility of greater than 100 g per 100 mL water at 25° C. Examples of porogens include carbohydrates (e.g., sorbitol, dextran (poly(dextrose)), starch), salts, sugar alcohols, natural polymers, synthetic polymers, and small molecules.

In certain embodiments, carbohydrates are used as porogens in composite or non-composite materials used to make the inventive bone anchors. The carbohydrate can be a monosaccharide, disaccharide, or polysaccharide. The carbohydrate can be a natural or synthetic carbohydrate. Preferably, the carbohydrate is a biocompatible, biodegradable carbohydrate. In certain embodiments, the carbohydrate is a polysaccharide. Exemplary polysaccharides include cellulose, starch, amylose, dextran, poly(dextrose), glycogen, etc. In certain embodiments, the polysaccharide is dextran. Very high molecular weight dextran has been found particularly useful as a porogen. For example, the molecular weight of the dextran can range from about 500,000 g/mol to about 10,000,000 g/mol, preferably from about 1,000,000 g/mol to about 3,000,000 g/mol. In certain embodiments, the dextran has a molecular weight of approximately 2,000,000 g/mol. Dextrans with a molecular weight higher than 10,000,000 g/mol can also be used as porogens. Dextran can be used in any form (e.g., particles, granules, fibers, elongated fibers) as a porogen. In certain embodiments, fibers or elongated fibers of dextran are used as the porogen in the composite or non-composite bone anchor. Fibers of dextran can be formed using any known method including extrusion and precipitation. Fibers can be prepared by precipitation by adding an aqueous solution of dextran (e.g., 5-25% dextran) to a less polar solvent such as a 90-100% alcohol (e.g., ethanol) solution. The dextran precipitates out in fibers that are particularly useful as porogens in the inventive bone anchors. Dextran can be about 15% by weight to about 30% by weight of the composite or non-composite material. In certain embodiments, dextran is about 15% by weight, 20% by weight, 25% by weight, or 30% by weight. Higher and lower percentages of dextran can also be used. Once the inventive anchor with the dextran as a porogen is implanted into a subject, the dextran dissolves away very quickly. Within approximately 24 hours, substantially all of the dextran is out of the material leaving behind pores in the implanted bone anchor. An advantage of using dextran is that it exhibits a hemostatic property in the extravascular space. Therefore, dextran in a bone anchor can decrease bleeding at or near the site of implantation.

Small molecules including pharmaceutical agents can also be used as porogens in the composite or non-composite bone anchors of the present invention. Examples of polymers that may be used as porogens include poly(vinyl pyrollidone), pullulan, poly(glycolide), poly(lactide), and poly(lactide-co-glycolide). Typically low molecular weight polymers are used as porogens. In certain embodiments, the porogen is poly(vinyl pyrrolidone) or a derivative thereof. In some embodiments, plasticizers that are removed faster than the surrounding composite or non-composite material can also be considered porogens.

In certain embodiments, the bone anchors of the present invention can include a wetting or lubricating agent. Suitable wetting agents include water, organic protic solvents, organic non-protic solvents, aqueous solutions such as physiological saline, concentrated saline solutions, sugar solutions, ionic solutions of any kind, and liquid polyhydroxy compounds such as glycerol, polyethylene glycol (PEG), polyvinyl alcohol (PVA), and glycerol esters, and mixtures of any of these. Biological fluids can also be used as wetting or lubricating agents. Examples of biological fluids that may be used with the inventive bone anchors include blood, lymph, plasma, serum, or marrow. Lubricating agents can include, for example, polyethylene glycol, which can be combined with the polymer and other components to reduce viscosity. Alternatively or in addition, particulate material used in making an anchor, or a formed anchor, can be coated with a polymer by sputtering, thermal evaporation, or other techniques known to those skilled in the art.

Additionally, composites or non-composites of the present invention can contain one or more biologically active molecules, including biomolecules, small molecules, and bioactive agents, to promote bone growth and connective tissue regeneration, and/or to accelerate healing. Examples of materials that can be incorporated include chemotactic factors, angiogenic factors, bone cell inducers and stimulators, including the general class of cytokines such as the TGF-β superfamily of bone growth factors, the family of bone morphogenic proteins, osteoinductors, and/or bone marrow or bone forming precursor cells, isolated using standard techniques. Sources and amounts of such materials that can be included are known to those skilled in the art.

In certain embodiments, the composite or non-composite used in preparing the inventive bone anchors includes antibiotics. The antibiotics can be bacteriocidial or bacteriostatic. Other anti-microbial agents can also be included in the material. For example, anti-viral agents, anti-protazoal agents, anti-parasitic agents, etc. may be include in the composite or non-composite. Other suitable biostatic/biocidal agents include antibiotics, povidone, sugars, and mixtures thereof.

Biologically active materials, including biomolecules, small molecules, and bioactive agents can also be combined with a polymer and/or particles used to make a composite or non-composite bone anchor to, for example, stimulate particular metabolic functions, recruit cells, or reduce inflammation. For example, nucleic acid vectors, including plasmids and viral vectors, that will be introduced into the patient's cells and cause the production of growth factors such as bone morphogenetic proteins may be included in the bone anchor material. Biologically active agents include, but are not limited to, antiviral agent, antimicrobial agent, antibiotic agent, amino acid, peptide, protein, glycoprotein, lipoprotein, antibody, steroidal compound, antibiotic, antimycotic, cytokine, vitamin, carbohydrate, lipid, extracellular matrix, extracellular matrix component, chemotherapeutic agent, cytotoxic agent, growth factor, anti-rejection agent, analgesic, anti-inflammatory agent, viral vector, protein synthesis co-factor, hormone, endocrine tissue, synthesizer, enzyme, polymer-cell scaffolding agent with parenchymal cells, angiogenic drug, collagen lattice, antigenic agent, cytoskeletal agent, stem cells, including stem cells derived from embryonic sources, adult tissues such as fat, bone marrow, human umbilical cord perivascular cells, endometrium/menstrual flow, etc., bone digester, antitumor agent, cellular attractant, fibronectin, growth hormone cellular attachment agent, immunosuppressant, nucleic acid, surface active agent, hydroxyapatite, and penetraction enhancer. Additional exemplary substances include chemotactic factors, angiogenic factors, analgesics, antibiotics, anti-inflammatory agents, bone morphogenic proteins, and other growth factors that promote cell-directed degradation or remodeling of a polymer within the composite or non-composite material and/or development of new tissue (e.g., bone). RNAi or other technologies can also be used to reduce the production of various factors.

To enhance biodegradation in vivo, materials comprising the inventive bone anchors can also include different enzymes. Examples of suitable enzymes or similar reagents are proteases or hydrolases with ester-hydrolyzing capabilities. Such enzymes include, but are not limited to, proteinase K, bromelaine, pronase E, cellulase, dextranase, elastase, plasmin streptokinase, trypsin, chymotrypsin, papain, chymopapain, collagenase, subtilisin, chlostridopeptidase A, ficin, carboxypeptidase A, pectinase, pectinesterase, an oxireductase, an oxidase, or the like. The inclusion of an appropriate amount of such a degradation enhancing agent can be used to regulate implant duration.

These added components need not be covalently bonded to a component of the material used to make an inventive bone anchor. An added component can be selectively distributed on or near the surface of the inventive bone anchor using the layering techniques described above, and e.g., spraying, dip coating, sputtering, thermal evaporation. While the surface of the anchor may be mixed somewhat as the anchor is manipulated in the implant site, the thickness of the surface layer will ensure that at least a portion of the surface layer remains at or near the surface of the inventive bone anchor. In some embodiments, biologically active components are covalently linked to the bone particles before combination with the polymer. For example, silane coupling agents having amine, carboxyl, hydroxyl, or mercapto groups can be attached to the bone particles through the silane and then to reactive groups on a biomolecule, small molecule, or bioactive agent.

The material comprising the bone anchor can be seeded with cells. In certain embodiments, a patient's own cells are obtained and used in preparing the composite or non-composite, from which an anchor is formed. Certain types of cells (e.g., osteoblasts, fibroblasts, stem cells, cells of the osteoblast lineage, etc.) can be selected for use in preparing the composite or non-composite. The cells can be harvested from marrow, blood, fat, bone, muscle, connective tissue, skin, or other tissues or organs. In certain embodiments, a patient's own cells are harvested, optionally selected, expanded, and used in the composite or non-composite. In other embodiments, a patient's cells are harvested, selected without expansion, and used in preparing the composite or non-composite. Alternatively, exogenous cells can be employed. Exemplary cells for use with the composite or non-composite include mesenchymal stem cells and connective tissue cells, including osteoblasts, osteoclasts, fibroblasts, preosteoblasts, and partially differentiated cells of the osteoblast lineage. The cells can be genetically engineered. For example, the cells can be engineered to produce a bone morphogenic protein.

In embodiments where the polymer component becomes formable when heated, the heat absorbed by particles in the composite or non-composite can increase the cooling time of the material, extending the time available to form the material into an anchor or adapt the anchor to an implant site. Depending on the relative heat capacities of the particle and the polymer components and the size of the particles, the particles may continue to release heat into the surrounding polymer after the time when the polymer alone would have cooled. The size and density distribution of particles within the composite can be optimized to adjust the amount of heat released into portions of an implanted bone anchor during and after implantation.

### Bone-Anchor Designs

In various embodiments, the inventive bone anchor is provided preformed in any of a variety of shapes and sizes with various features. For example, the bone-anchor shapes can include rods, cylinders, tapered cylinders, cones, rectangles, cubes, oval cylinders, partial cylindrical strips, tubes, polygonal tubes, and pyramids. In some embodiments, the inventive bone anchors are tulip shaped. The sizes of the bone anchor can include outer diameters of any value between about 5 millimeters (mm) to about 50 millimeters, and lengths of any value between about 5 millimeters to about 75 millimeters. In some embodiments, the outer diameter of the inventive bone anchor is between about 5 mm and about 10 mm, between about 10 mm and about 15 mm, between about 15 mm and about 20 mm, between about 20 mm and about 30 mm, between about 30 mm and about 40 mm, and yet between about 40 mm and about 50 mm. In some embodiments, the length of the inventive bone anchor is between about 5 mm and about 10 mm, between about 10 mm and about 15 mm, between about 15 mm and about 20 mm, between about 20 mm and about 30 mm, between about 30 mm and about 40 mm, between about 40 mm and about 50 mm, between about 50 mm and about 60 mm, and yet between about 60 mm and about 75 mm. A particular shape and size can be selected based upon the dimensions of a void at the implant site. The features of the anchor can include threads, ridges, grooves, slots, latching rims, protrusions, bumps, barbs disposed on the outer and/or inner surfaces of the anchor and various head designs, e.g. pan head, flanged head, slotted head, hexagonal head, square head, and no head. In various embodiments, an inventive bone anchor comprises a hollow core, one or more slits or divisions extending longitudinally along at least a portion of the anchor, wherein at least a portion of the bone anchor can expand radially outward upon insertion of a screw or fastening device into the hollow core. In some embodiments, the bone anchor has no slits or divisions extending longitudinally along the anchor.

In some embodiments, the bone anchor is provided as a mass of material which can be formed into a shape suitable for placement in bone at a site of surgical intervention. As an example, the bone anchor comprises Plexur M™ material provided by Osteotech, Inc. of Eatontown, N.J. In various aspects, the material can be made moldable and packed into a void in the bone. The material can then be hardened, and subsequently drilled, reamed, cut, ground, threaded, or any combination thereof.

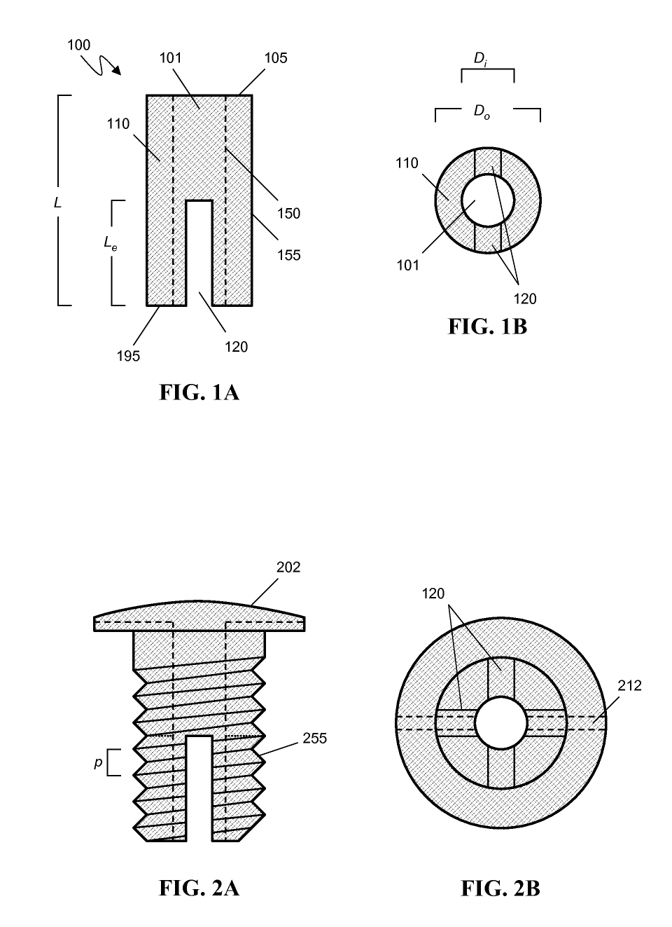

Referring now to FIG. 1, an embodiment of a bone anchor 100 is depicted in elevation view (1A) and bottom view (1B). The bone anchor comprises and elongate element adapted for placement within a void in a bone, and is also adapted to receive and secure a fastening device. The anchor has a length L, and is substantially cylindrical in shape with a hollow core 101. The embodied anchor 100 has a near end 105 and a distal end 195, and slots 120 are incorporated into the distal end of the anchor's wall 110 extending length Lₑ along the length of the anchor. The bone anchor can be tubular in shape and have an inner diameter Dᵢ and inner surface 150, and outer diameter Dₒ and outer surface 155. For the embodiment shown in FIGS. 1A-1B, both Dᵢ and Dₒ are substantially constant along the length of the anchor. The length of the anchor L can be in a range between about 3 millimeters (mm) and about 5 mm, between about 5 mm and about 10 mm, between about 10 mm and about 20 mm, between about 20 mm and about 40 mm, and yet between about 40 mm and about 80 mm in some embodiments. The maximum outer diameter Dₒ of the anchor can be in a range between about 5 mm and about 10 mm, and the maximum inner diameter Dᵢ of the anchor can be in a range between about 2 mm and about 8 mm. The maximum outer diameter of the anchor can be in a range between about 10 mm and about 20 mm, and the maximum inner diameter of the anchor can be in a range between about 8 mm and about 17 mm. In some embodiments for primary placement of an inventive bone anchor, the anchor has an outer diameter of about 6 mm and a length of about 5 mm. In some embodiments for surgical revision procedures, the anchor has an outer diameter in a range between about 9 mm and about 11 mm, and a length in a range between about 6 mm and about 7 mm.

In certain embodiments, the inner surface 150 incorporates threads, ribbing, ridges, grooves, or other protrusions or indentations providing features for inserting and securing or attaching a fastening device to the anchor 100. In various embodiments, the fastening device can be a screw, pin, rod, bolt, spring pin, rivet-like pin, or the like. In some embodiments, the fastening device includes one or more longitudinally-oriented holes or grooves, and the one or more holes or grooves is adapted to accommodate a guide wire, rod or pin to aid in placement of the fastening device. The fastening device can include mating threads, ribs, ridges, grooves, or the like to improve its securing within the bone anchor. Additionally, the outer surface 155 of the anchor can incorporate threads, ribbing, ridges, grooves, or other protrusions or indentations to facilitate secure placement of the anchor within an implant site. In certain embodiments, the outer surface 155 is treated with a bioactive material, e.g., hydroxyapatite, which promotes growth of bone up to the implant and into the implant. In certain embodiments, the slots 120 extend about one-quarter, between about one-quarter and about one-half, about one-half, between about one-half and three-quarters, about three quarters, or greater than three-quarters along the length of the anchor. There can be one, two, three, four, five or six slots 120 incorporated into the anchor's wall 110.

The bone anchor can incorporate a variety of shape features. For example, the inner diameter Dᵢ of the anchor can vary, continuously or in a step-wise manner, along the length of the anchor. For example, it can be smaller at the distal end 195 than at the near end 105, e.g., as depicted in FIG. 4A. The outer diameter Dₒ of the anchor can vary, continuously or in a step-wise manner, along the length of the anchor. For example, it can be smaller at the distal end 195 than at the near end 105 in some embodiments, and larger at the distal end 195 than at the near end 105 in some embodiments.

The slots 120 in the wall 110 of the anchor 100 readily permit outward radial expansion of the portion of the anchor incorporating the slots. In some embodiments, the depth of the slots are less than the thickness of the anchor wall, so that the slots do not extend through the anchor wall. In some embodiments, the anchor is malleable and has no slots. In various embodiments, insertion of a fastening device into the core of the anchor 101 forces the walls radially outward and into intimate contact with surrounding native bone. For example, the diameter of the fastening device can be slightly larger than Dᵢ, or the fastening device can have a gradually increasing diameter along its length, varying from a value slightly less than Dᵢ to a value slightly greater than Dᵢ, or the anchor 100 can have a smaller inner diameter Dᵢ at its distal end 195 than at its near end 105. Upon full insertion of the fastening device, the outer walls along the slotted portion are forced outwards. In this manner, the inventive bone anchor may function like plastic expansion anchors. The outward radial expansion of a portion of the anchor can provide resistance against pull-out of the anchor by increasing the contact area between the host implantation site and the anchor. In some embodiments, the outward radial expansion and malleable material properties of the anchor allow the anchor to conform and fill uneven and/or non-uniform geometries and surface features of the host implantation site.

In some embodiments, the inventive bone anchors expand upon hydration. As an example, a bone/polymer or bone/substitute polymer composite from which the anchor is formed can absorb water or biological fluids. The water or fluids can be adsorbed into the matrix of the bone/polymer or bone/substitute polymer composite. In certain embodiments, the adsorption increases the volume of the composite and causes an expansion of the anchor's outer diameter. The expansion upon hydration can provide securing of the anchor in a void, e.g., by forcing at least a portion of the anchor into intimate contact with surrounding bone.

The bone anchor can be preformed and made available in an array of sizes, or the anchor can be formed immediately prior to implantation. The anchor can be inserted in a natural or prepared void in native bone. For example, the anchor can be placed in a void that has been prepared by drilling and optionally tapping threads into the bone.

In certain embodiments, the bone anchor is formed from a composite, as described above, which can undergo a phase-state transition. The phase state transition can be from a formable, moldable, pliable, or flowable state to a substantially solid state or rigid state. The phase transition can be reversible such that the composite can be transformed from a substatianlly solid state to a formable, moldable, pliable, or flowable state and back to a substantially solid state. In certain embodiments, the transformation occurs within biocompatible temperature ranges or biocompatible chemical conditions.

In certain embodiments, the bone anchor is made malleable by heating or adding a solvent to the composite. The anchor can then be placed into an implantation site (e.g., a bony defect) followed by setting of the composite. The composite can be set by allowing the composite to come to body temperature, increasing the molecular weight of the polymer in the composite, cross-linking the polymer in the composite, irradiating the composite with UV radiation, adding a chemical agent to the polymer, or allowing a solvent to diffuse from the composite. The solidified bone anchor can be allowed to remain at the site providing the strength desired while at the same time promoting healing of the bone and/or bone growth.

The polymer component of the composite can degrade or be resorbed as new bone is formed at the implantation site. The polymer can be resorbed over approximately 1 month to approximately 6 years. In some embodiments, the polymer is resorbed over an amount of time between about 1 month and about 3 months, between about 3 months and about 6 months, between about 6 months and about 12 months, between about 12 months and about 18 months, between about 1 year and about 2 years, between about 2 years and about 3 years, between about 3 years and about 4 years, between about 4 years and about 5 years, and yet between about 5 years and about 6 years. The anchor can start to be remodeled, i.e., replaced with new cell-containing host bone tissue, in as little as a week as the composite is infiltrated with cells or new bone in-growth. The remodeling process may continue for weeks, months, or years.

FIGS. 2A-2B depict an embodiment of a bone anchor having a head 202 and threads 255 of pitch p. FIG. 2B is a bottom-up view of the anchor. The threads are formed on the outer surface of the anchor, such that the anchor can be screwed into an implant site. The embodied anchor has four slots 120 and a slot 212 extending across the head 202 of the anchor. A screwdriver or similar torque-inducing mechanism can be inserted into slot 212 to assist in insertion of the anchor at the implant site. A pan-head style is depicted for the anchor shown in FIGS. 2A-2B, although other head styles can be used, e.g., round-head, oval-head, flat-head, bullet-head, hexagonal head, socket-head, etc. In some embodiments, the anchor can have no outwardly flanged head. In some embodiments, the lower slotted portion of the anchor expands radially outward upon insertion of a screw or fastening device. The outward radial expansion of a portion of the anchor can provide resistance to pull-out of the anchor.

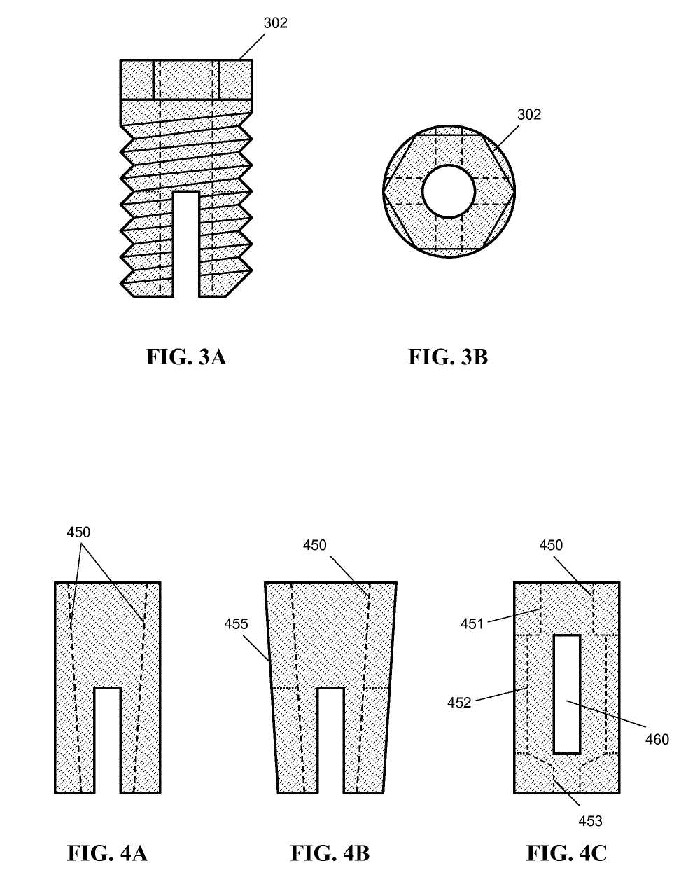

An embodiment of an anchor having a hexagonal head 302 is shown in FIGS. 3A-3B. A top-down view is shown in FIG. 3B. For this embodiment, any of a variety of wrench types, e.g., adjustable, box-end, socket, 12-point, is used to apply torque to the anchor during insertion at an implant site.

Although the embodiments of FIG. 2 and FIG. 3 depict uniform-pitch p threading along a substantially constant outer diameter surface of the anchor, other embodiments incorporate varied-pitch threading and/or tapered outer diameter surfaces. Varied-pitch threading and/or a tapered outer diameter can facilitate binding of the anchor within the implant site as the anchor is tightened within the site. In yet other embodiments, the outer surface is ribbed, e.g., having multiple parallel ridges running around the circumference of the cylinder. In yet other embodiments, the outer surface has one or more grooves or ridges running longitudinally along the surface of the cylinder. The grooves or ridges can run substantially straight along the outer surface, or can run along the surface with a slight helical trajectory. The longitudinal grooves or ridges can prevent the anchor from rotating in the implantation site. In some embodiments, the anchor comprises a combination of threads and grooves or ridges, or a combination of ribbed structure and grooves or ridges.

FIGS. 4A-4C depict embodiments of bone anchors having various features. For any of the depicted embodiments, including those of FIGS. 1-6 and FIG. 8, at least a portion of the outer surface and all, or a portion of the inner surface can incorporate threads, ribbing, grooves, ridges, barbs, or other features to improve gripping of the anchor to surrounding bone and of a fastening device to the anchor. In FIG. 4A the inner diameter Dᵢ varies continuously from a value at the near end to a smaller value at the distal end. The resulting inner surface 450 is conical in shape. The fastening device can also have a complementary conical or tapered shape. In FIG. 4B both the inner and outer diameters taper to smaller values at the distal end of the anchor, giving a conical shape to the inner 450 and outer 455 surfaces. In certain embodiments, an anchor shaped substantially as shown in FIG. 4B is used for placement of a pedicle screw into a pedicle. In certain embodiments, the anchor is made of a composite material comprising bone or a bone substitute and a polymer (e.g., PLGA, PLA, PGA, polyesters, polycaprolactone, polyurethanes, etc.). In certain embodiments, the anchor is preformed from Plexur P™ material provided by Osteotech, Inc. of Eatontown, N.J. In certain embodiments, the anchor is made from a material described in one of the patents or patent applications incorporated herein by reference. For either embodiment shown in FIGS. 4A-4B, a fastening device having a uniform diameter or tapered diameter can force the walls along the slotted portion at the distal end radially outward upon full insertion.

In FIG. 4C, the inner diameter varies in a step-wise manner. A portion of the anchor 451 at the near end has an inner diameter of a first value. This diameter can be large enough so that a threaded fastening device slips through. A portion of the anchor 452 has an inner diameter of a second value larger than the first value. A portion of the anchor 453 has an inner diameter of a third value, which can be small enough to engage the threads of an inserted fastening device. Slots 460 are incorporated in the anchor along portion 452 where the walls have the thinnest dimension. In certain embodiments, a fastening device engages a threaded portion 453 when inserted, and when tightened acts to compress the anchor along its length. The compressive action forces the walls along portion 452 radially outward and into intimate contact with surrounding native bone. In certain aspects, the bone anchor depicted in FIG. 4C functions similarly to a molly bolt anchor or sleeve-type hollow wall anchor.

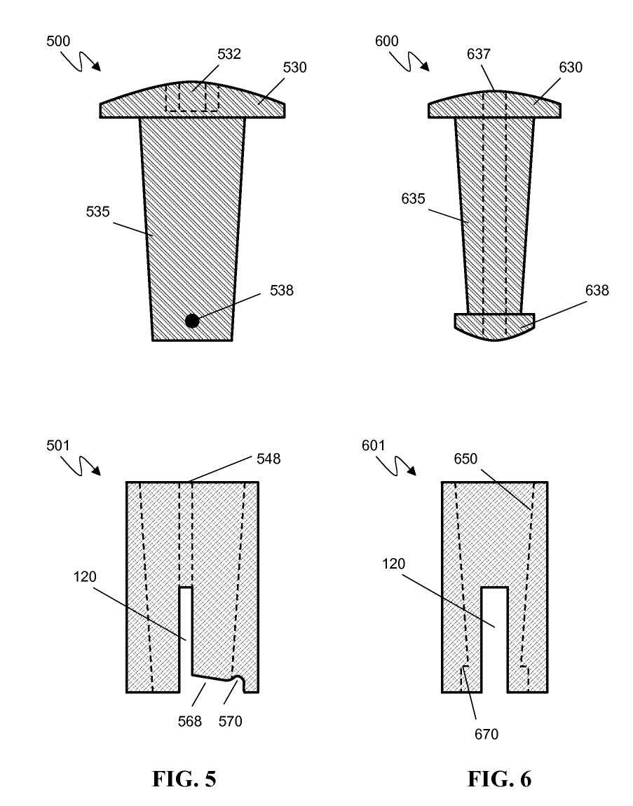

An inventive bone anchor can be adapted to receive a bayonet-type fastening device, wherein the bayonet fastening device is rotatable to a locked position upon insertion. An embodiment of a bone anchor 501 and fastening device 500 having features to provide bayonet-type fastening is depicted in FIG. 5. The anchor 501 incorporates at least one slot 120 at its distal end. The slot 120 opens circumferentially at the distal end having a sloping profile 568 and indent 570. The anchor's inner surface incorporates a groove 548, substantially aligned with the slot. The anchor's inner diameter is gradually reducing from its near end to its distal end, and its conical shape substantially matches that for the shaft 535 of the fastening device 500. A pin 538 extends through the shaft 535 of the fastening device, and is accepted into the groove 548 of the anchor upon insertion. The fastening device can be provided with a head 530 as shown, and the head can have a hex-socket recess 532 for the insertion of a torque-applying tool. Upon substantially full insertion of the fastening device 500 into the anchor 501, the pin 538 passes along the groove 548 and slot 120 to a position about adjacent to the sloping profile 568. At this point, a torque-applying tool can be inserted into the recess 532 and the fastening device 500 rotated such that the pin 538 engages the sloping profile 568. Further rotation can draw the fastening device downwards, expand the walls of the anchor radially outward at the distal end, and move the pin to the detent 570 whereupon the fastening device becomes substantially locked in position.

The inventive bone anchor can be adapted to receive a latching rivet-like fastening device, wherein the fastening device can be tapped, pressed or driven into a locked position within the anchor. In certain embodiments as depicted in FIG. 6, the bone anchor 601 and fastening device 600 can incorporate features to provide latching, rivet-like operation. The anchor 601 has an inner surface that is substantially conical in shape, and incorporates one or more slots 120 at its distal end. Additionally, a flanged rim 670 is provided at the distal end on the inner surface. The fastening device 600 has a tapered shaft 635 that substantially matches the shape of the anchor's inner surface. The fastening device includes a near-end head 630 and a distal foot 638. The outer diameter of the foot is small enough in value to allow insertion into the near end of the anchor, but larger in value than the inner diameter of the distal end of the anchor. Upon insertion, the fastening device 600 is pressed or tapped down into the anchor 601. When tapped in, the foot 638 forces the walls at the distal end radially outward, improving their contact with the surrounding native bone. When fully inserted, the foot 638 latches over the flanged rim 670 substantially locking the fastening device 600 in the anchor 601.

In some embodiments, the shafts 535, 635 of the fastening devices 500, 600 has one or more grooves running longitudinally along their outer surface, or has one or more holes 637 running longitudinally through the fastener. The one or more holes need not be central to the shaft. In certain embodiments, the one or more grooves or one or more holes accommodate one or more guide wires or pins. As an example and referring to FIG. 6, a guide wire or pin can be placed substantially centrally in a prepared void in a bone. An anchor 601 can be guided to the implant site by first threading the guide wire or pin centrally through the anchor. The anchor can then be guided to the implant site by sliding it along the guide wire or pin. Once the anchor 601 is placed, a fastener 600 can be guided to the anchor in a similar manner. The guide wire, rod, or pin can be subsequently removed.

In certain embodiments, a bone anchor as depicted in any of FIGS. 1-6 is provided in pieces, which together form an anchor. For example, any of the depicted anchors can be halved or quartered along their axis of symmetry, and each of the pieces can be inserted sequentially into an implant site.

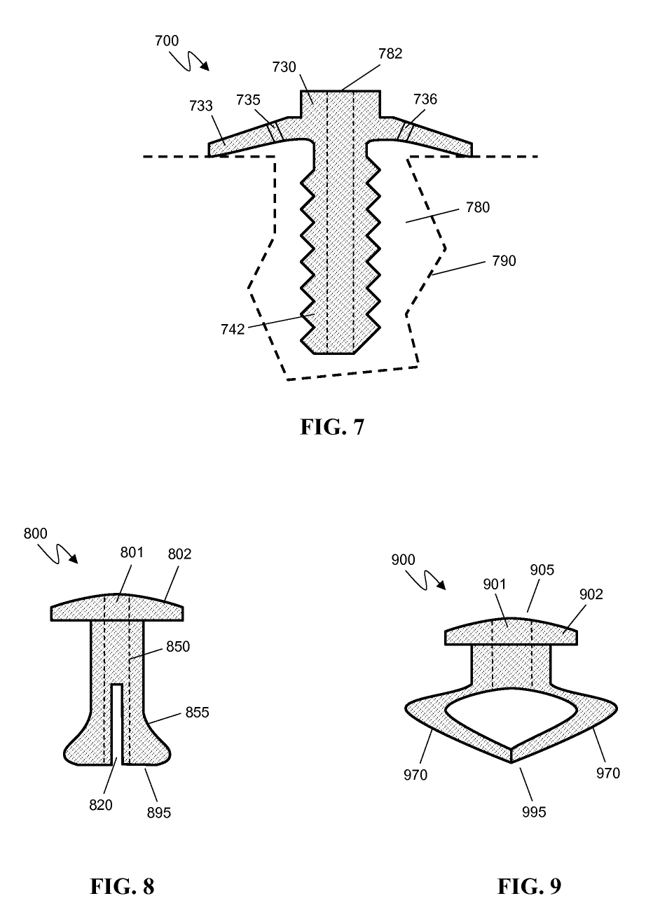

In various embodiments, a bone anchor is formed in situ or in vivo. FIG. 7 depicts, in cross-section elevation view, a fastening-device form 700 useful for forming a bone anchor in situ of in vivo. The fastening-device form generally replicates a fastening device, but can be made from or incorporate a separate material that minimally sticks to the solidified bone-anchor composite. For, example the form 700 can be made from polytetrafluoroethylene (PTFE or Teflon) or incorporate a Teflon or fluoro-polymer coating on its shaft 742. In some embodiments, the form 700 can be made from a polished metal. The fastening-device form 700 can include a threaded, grooved, ridged, or smooth shaft 742, a head 730 and a semi-flexible flange or gasket 733. In some embodiments, the fastening-device form 700 has one or more holes running longitudinally through its shaft 742 or one or more grooves oriented longitudinally on the outer surface of the shaft 742 to accommodate one or more guide wires or pins and to aid in placement of the form 700 at the implant site. The flange or gasket can incorporate one or two holes 735, 736 extending through the gasket. In use, the form 700 can be placed substantially centrally in an implant site, such as a void in a bone indicated by the dashed line 790, and held firmly in place. The void can be irregular in shape as depicted. Flowable bone/polymer or bone substitute/polymer composite can then be injected through hole 736 filling the vacancy between the form 700 and the surrounding bone 790. In some embodiments, the injection can be performed using a cannula, e.g., a cannula having a 3-mm-diameter bore. The vacancy will be filled when composite emerges from under the gasket or through hole 735. When the composite solidifies, the form 700 can be removed, for example, by placing a torque-applying tool on head 730 and unscrewing or extracting the form out of the implant site. Subsequently, a fastening device can be placed in the vacancy remaining after extraction of the form 700. In some embodiments, a fastening device is used directly at the implant site, instead of form 700, eliminating the requirement of removing the form 700 after composite solidification.

In some embodiments, a metal form 700 provides a higher heat capacity than a similarly shaped Teflon form, and can provide more rapid cooling of heated composite. A metal form can be coated with a fluoro-polymer to reduce adhesion between the form 700 and cooled composite.

In some embodiments, a cannula and form 700 are adapted to provide functionality for both guiding the form 700 to the implant site and filling the vacancy 780 with composite. For example, a cannula can be positioned with one end in the bony defect. A form 700 can be place onto the cannula by threading the cannula through a longitudinal hole 782 running through the form 700. The form 700 can then be guided down into the bony defect via the cannula. A supply of flowable composite can then be attached to the cannula. Flowable composite can then be delivered to the bony defect via the cannula. In an alternative embodiment, the form 700 can be threaded onto the cannula, with supply of composite attached to the cannula, before one end of the cannula is positioned in the bony defect.

An embodiment of a tulip-shaped bone anchor 800 is depicted in FIG. 8. For this, and similar embodiments, the distal end 895 of the anchor 800 is flared outwards, and contains slots 820. The outward flare of the anchor's distal end 895 can provide resistance against pull-out of the anchor. There can be one, two, three, four, or more slots 820 in the distal end 895, and these slots can provide for radial-outward expansion of the anchor's distal end upon insertion of a screw or fastening device into the anchor's central core 801. The tulip-shaped anchor 800 can include a head 802 at its near end in some embodiments, or can not include a head in some embodiments. In some embodiments, the tulip-shaped anchor 800 includes threads, ribbing, ridges, or grooves, or any combination thereof, on its outer 855 and/or inner 850 surfaces.

An embodiment of a winged anchor 900 is depicted in FIG. 9. The winged anchor 900 comprises two wings 970 at its distal end 995. Prior to inserting the anchor 900 into a hole or void, the wings 970 can be folded toward each other, so that they slip through a hole. After insertion, the wings 970 can be spread apart, and a screw or fastening device can be inserted into the anchor's hollow core 901. Insertion of a screw can engage the wings 970 drawing them back toward the near end 905 of the anchor. The wings 970 can provide resistance against pull-out of the anchor. In some embodiments, the winged anchor 900 has a head 902, and in some embodiments the anchor can be provided without a head.

In certain embodiments, the inventive bone anchors, e.g., any anchor depicted in FIGS. 1-9, are adapted to accommodate a support wire or rod. The support wire or rod can provide additional strength at the implant site. An accommodation for a support wire or rod can include a groove or hole as part of the bone anchor's form. For example in some embodiments, an accommodating groove runs longitudinally along an inner or outer surface of the anchor, or across the head of the anchor. In some embodiments, an accommodating hole runs longitudinally through the anchor body or the anchor head, or runs transverse through the anchor body or anchor head. In certain embodiments, the method includes preparing the site to receive the bone anchor.

In certain embodiments, the inventive bone anchor is preformed into an anchor-like shape prior to placement in a void in a bone. The preformed shape can be any shape depicted in FIGS. 1-9, or similar shapes suitable for anchoring a fastening device. In some embodiments, the preformed anchor comprises a bone/polymer composite. In some embodiments, the preformed anchor comprises a bone substitute/polymer composite. In certain embodiments, Plexur P™ material, e.g., an osteoconductive biocomposite of cortical fibers suspended in a resorbable, porous polylactide-co-glycolide scaffold, containing calcium, phosphate, trace elements and extracellular matrix proteins which promote bone healing, provided by Osteotech, Inc. of Eatontown, N.J., or Plexur M™ material also provided by Osteotech, Inc. is used to make the preformed bone anchor. Preformed bone anchors can be provided in an array of sizes and shapes to cover a range of placement sites in bones. For example, after evaluating a placement site in a bone, an attending physician can select from a group of bone anchors one preformed bone anchor which is deemed suitable or most suitable for the placement site.

In certain embodiments, the inventive bone anchor is not preformed. Rather, the bone anchor can be moldable, or made moldable, for placement at a placement site in bone. A non-preformed anchor may not have particular features as depicted in FIGS. 1-9. A non-preformed anchor can be provided as a solid mass of bone/polymer or bone substitute/polymer material. In certain embodiments, Plexur P™ material provided by Osteotech, Inc., or Plexur M™ material also provided by Osteotech, Inc. is used to make the non-preformed bone anchor. A non-preformed anchor can be provided substantially as a cylinder of material, e.g., a solid cylinder of material, a tapered cylinder, an elliptically shaped cylinder, an oblong sphere, a cored dowel, or as a cube, rectangular block, or sphere of material. In some embodiments, non-preformed anchors are provided in an array of sizes and shapes to cover a range of placement sites in bones. After evaluating a placement site in a bone, an attending physician can select from a group of non-preformed bone anchors one which is deemed suitable or most suitable for adaptation to the placement site. In certain embodiments, a non-preformed anchor is provided as a moldable substance, which can be solidified after placement in bone. In some embodiments, a non-preformed anchor is provided as a substantially solid or semi-solid mass, which can be made moldable by the application of heat or an additive. When moldable, the non-preformed anchor can be shaped by an attending physician or clinician for placement in a bone placement shape. In various embodiments, a non-preformed anchor can be adapted for placement in a bone placement site by an attending physician, e.g., by molding, pressing, carving, cutting, grinding, drilling, threading, reaming, and any combination thereof.

In some embodiments, bone particles and/or particles of a bone substitute material are combined with a polymer, mixed and substantially solidified in a manner to form a bone anchor having a concentration or density gradient. In certain embodiments, a flowable composite can be introduced into a mold. The composite in the mold can be subjected to an electric field which redistributes particles within the composite and the composite subsequently solidified. In some embodiments, a flowable composite can be introduced into an electromagnetically-transparent mold. A spatially-varying dose of radiation, e.g., ultraviolet radiation, infrared radiation, microwave radiation, can be applied to the composite to spatially selectively solidify or alter the density of composite as it transforms to a substantially solid state.

### Methods of Using Inventive Bone Anchors

In one aspect, the invention includes methods of using the inventive bone anchors in various surgical procedures. The methods are useful in orthopedic surgery and dentistry, and can be particularly useful in spinal surgery or skeletal surgery. The inventive bone anchors can be used in methods for placement of pedicle screws, e.g., in such procedures as interbody fusion (IBF), anterior lumbar interbody fusion (ALIF), etc. In various embodiments, the methods disclosed herein are particularly useful for surgical procedures in which the patient presents osteoporotic bone, diseased bone, bony defects, bone tumors, bone that has undergone traumatic injury, previous skeletal surgery, or previous joint replacement.

In spinal surgery applications, an inventive bone anchor can be placed in the pedicles or the body of vertebrae. The pedicle or vertebral body can be normal, osteoporotic, or diseased bone. In some embodiments, an inventive bone anchor is placed in the spinous or transverse process of vertebrae. The spinous or transverse process of vertebrae can be normal, osteoporotic, or diseased bone. In certain embodiments, an inventive bone anchor is placed in the sacrum. The sacrum can be normal, osteoporotic, or diseased bone.

In some embodiments, an inventive bone anchor is placed in cancellous regions of long bones. In some embodiments, an inventive bone anchor is placed in normal, osteoporotic or diseased regions of long bones. In some embodiments, an inventive anchor is placed in cancellous regions of long bones where the tissue is normal, osteoporotic or diseased. In yet additional embodiments, an inventive bone anchor is placed in cortical regions of various bones. In various embodiments, the methods include providing an inventive bone anchor, and placing the bone anchor in a void at an implant site.

In certain embodiments, a method of placing an inventive bone anchor comprises implanting the bone anchor into a void in the pedicle or the body of a vertebra or sacrum of a subject, and securing a fastening device into the bone anchor. The method can further include implanting a bone anchor in multiple vertebrae of a subject. In some embodiments, the method of placing an inventive bone anchor includes molding or adapting the shape of the anchor to conform to or fit within a void in a vertebra or sacrum.

The void at an implant site can be a natural void, a defect, a wound, or a prepared void in a bone. A natural void, defect or wound can be in the shape of a depression, divot, or hole in a bone. A prepared void can be formed by drilling, reaming, cutting, or grinding processes, or any combination thereof, to remove an amount of bone. A prepared void could include forming threads, ridges, ribs or grooves in the bone to mate with similar features on a bone anchor to be placed in the void. In some embodiments, the void comprises missing or underdeveloped bone, a defect, or a removed defect such as a tumor or spur. The bone anchor can include additional material to span across an area of the bone or wrap around a portion of the bone. In various embodiments, the void is located in a bone having a characteristic selected from the following group: normal, cancellous, diseased, and osteoporotic.

An inventive bone anchor can be administered to or placed in a subject in need thereof using any technique known in the art. In various embodiments, an inventive bone anchor can be inserted into an implant site. The subject is typically a patient with a disorder or disease related to bone. In certain embodiments, the subject has a bone or joint disease typically involving the spine. In some embodiments, the subject presents a skeletal disorder in non-spinal bones. In certain embodiments, the subject has a disease which includes bony defects, e.g., bone metastises. The subject is typically a mammal although any animal with bones can benefit from treatment with an inventive anchor. In certain embodiments, the subject is a vertebrate (e.g., mammals, reptiles, fish, birds, etc.). In certain embodiments, the subject is a human. In other embodiments, the subject is a domesticated animal such as a dog, cat, horse, etc.

Any bone disease or disorder can be treated using the inventive bone anchors including genetic diseases, congenital abnormalities, fractures, iatrogenic defects, bone cancer, trauma to the bone, surgically created defects or damage to the bone which need revision, bone metastases, inflammatory diseases (e.g. rheumatoid arthritis), autoimmune diseases, metabolic diseases, and degenerative bone disease (e.g., osteoarthritis). In certain embodiments, an inventive bone anchor is formed or selected for the repair of a simple fracture, compound fracture, or non-union; as part of an external fixation device or internal fixation device; for joint reconstruction, arthrodesis, arthroplasty; for repair of the vertebral column, spinal fusion or internal vertebral fixation; for tumor surgery; for deficit filling; for discectomy; for laminectomy; for excision of spinal tumors; for an anterior cervical or thoracic operation; for the repairs of a spinal injury; for scoliosis, for lordosis or kyphosis treatment; for intermaxillary fixation of a fracture; for mentoplasty; for temporomandibular joint replacement; for alveolar ridge augmentation and reconstruction; as an inlay osteoimplant; for implant placement and revision; for revision surgery of a total joint arthroplasty; for staged reconstruction surgery; and for the repair or replacement of the cervical vertebra, thoracic vertebra, lumbar vertebra, and sacrum; and for the attachment of a screw or other component to osteoporotic bone. Additional uses for the inventive bone anchors include reinforcing an anchoring site for the attachment of components of a spinal stabilization system, providing stabilization of the spine for spinal fusion procedures, including posterior lumbar interbody fusion (PLIF), anterior lumbar interbody fusion (ALIF), transforaminal lumbar interbody fusion (TLIF), other interbody fusion procedures in the lumbar, thoracic or cervical spine, posterolateral fusion in the cervical, thoracic or lumbar spine, treatment of osteoporotic or traumatic compression fractures of the vertebrae, adult spinal deformity correction, pediatric spinal deformity correction (scoliosis), etc.

A method of administering an inventive bone anchor can comprise the steps of (a) providing a suitable bone anchor for placement at an implant or placement site, and (b) placing the bone anchor in a void at the implant site. The step (a) of providing a suitable bone anchor can comprise assessing the implant site and selecting an anchor or anchor material based upon size, diameter, shape and depth of the placement site. Step (a) can further comprise selecting the anchor or material based upon strength and durability of the composite material, firmness and stability of fit of the bone anchor within the implant site, the bone anchor's ability to be rendered into a malleable or flowable state, or its ability to be solidified after placement. Step (a) can further comprise selecting an inventive bone anchor based upon the condition of the native bone at the placement site. The step (a) of providing a suitable bone anchor can be carried out in a clinical setting during surgical intervention. In step (a), an inventive bone anchor can be provided in solid form, malleable form, or liquid form. Step (a) can include molding a bone anchor in a shape suitable for the placement site. Step (b) of placing the bone anchor in a void at the placement site can include inserting the anchor into the site via injecting, pressing, tamping, tapping, screwing, piece-wise inserting and the like. In various embodiments, injecting of an anchor is carried out using a cannula. In certain embodiments, a cannula is used with an orifice of about 1 mm, 2 mm, 3 mm, 4 mm, 5 mm or larger diameter. Step (b) can include using one or more guide wires, rods, cannulas or pins to guide the bone anchor and/or fastening device to the implant site. In some embodiments, the guiding device is the cannula. Step (b) can also include rendering the bone-anchor composite in a flowable or malleable state, injecting the flowable bone-anchor composite, and/or drilling the bone-anchor composite after implantation. Step (b) can further comprise solidifying the bone anchor after implantation. Step (b) can also include modifying the shape of the bone anchor, e.g., by carving, sanding, or grinding, so that it can be received by the implant site. In certain embodiments, step (b) can include providing a fastening-device form at the implant site, and injecting flowable bone/polymer or bone substitute/polymer composite within and/or around the form. Step (b) can include solidifying the bone-anchor composite after placement. In certain embodiments, additional steps of administering an inventive bone anchor optionally include (c) assessing in-growth of native bone, or assessing replacement or resorption of at least a portion of an inventive bone anchor, (d) inserting a fastening device into the bone anchor after implantation, (e) adapting the implant site to receive the bone anchor, and (f) attaching a prosthetic to a portion of the bone anchor or to a fastening device attached to the bone anchor. The step (e) of adapting the implant site can include drilling, reaming, cutting, grinding, and/or threading the placement site so that it can receive an inventive bone anchor. In certain embodiments, the steps of administering an inventive bone anchor can be performed on a patient at widely separated points in time, e.g., as may occur in staged surgery. As an example of staged surgery, one or more inventive bone anchors can be placed during a first surgical intervention. The one or more anchors can be placed and secured at distal fixation points. Screws or fastening devices can be placed with the one or more anchors during the first surgical intervention. In subsequent surgery, hardware necessary to correct a local deformity can be placed and affixed to the inventive anchors. It will be appreciated by one skilled in the art that any combination of steps (a)-(f), and subsidiary steps, described above can be used in administering an inventive bone anchor.

As an example of one of many methods enabled by the above steps, a method for administering an inventive bone anchor comprises (i) selecting a bone-anchor composite suitable for use at the implant site; (ii) rendering the composite into a flowable state; (iii) injecting the flowable composite into the implant site, where the injection can be done using a cannula; and (iv) forming a hole in the composite within the implant site. In some embodiments, the hole is formed by drilling the composite. In some embodiments, a hole is formed in the composite by placing a pin in the composite prior to solidification of the composite and extracting the pin after the composite solidifies. The pin can be coated with an anti-sticking chemical agent. Subsequently, screw or fastener can be placed in the hole.

As an example of another method, a method for administering an inventive bone anchor can comprise (i) preparing a hole in normal or osteoporotic bone, e.g., by drilling; (ii) placing a guide pin or wire in the hole; and (iii) placing an inventive bone anchor over the pin or wire, e.g., threading the anchor over the pin or wire, and guiding the anchor to the implant site with the guide pin or wire. In some embodiments, the method further includes (iv) removing the pin or wire; and (v) inserting a fastening device into the anchor. In some embodiments, alternative steps (iv) and (v) include (iv) placing a fastening device over the pin or wire, e.g., threading the fastening device over the pin or wire, and guiding the fastening device to the anchor; (v) inserting the fastening device in the anchor, (vi) removing the guiding pin or wire.

An embodiment of a procedure for placing an inventive bone anchor comprises optionally preparing a hole, e.g., by drilling or reaming; optionally placing a guide pin or guide wire in the hole; introducing an inventive bone anchor over the pin or wire; placing the anchor in the implantation site with the aid of the guide wire or pin, e.g., sliding it along the wire or pin into the prepared hole, removing the pin or wire; and placing a screw or other type of fastener into the anchor. In some embodiments, the screw or other type of fastener has a hole extending longitudinally through its shaft such that the screw or fastener can also be introduced into the anchor over the guide wire or pin prior to removal of the guide wire or pin.

Additional applications for the inventive bone anchors include their use in various dental procedures. As an example, an inventive bone anchor can be used to place prosthetic tooth implants. In such applications, the bone anchor can provide a secure attachment site for a tooth implant requiring a screw attachment. A tooth implant procedure can be carried out in several staged steps, and include the steps of preparing the implantation site, placing an inventive bone anchor at the implantation site, allowing for growth of bone at the implant site, placing a screw in the anchor, attaching a tooth implant to the screw. In some embodiments, a screw or fastening device is placed in the anchor prior to placement of the anchor at the site.

An inventive bone anchor is typically administered to a patient in a clinical setting. In certain embodiments, a bone anchor is administered during a surgical procedure. A bone anchor can be placed at an implant site by pressing, tapping, or screwing it into place. In some embodiments, the implant site is drilled and tapped to provide threads for screwing a bone anchor into the native bone. In some embodiments, a bone anchor can be approximately formed to fit in a void at the implant site by carving portions from the bone anchor and cutting or trimming its length with a scalpel or other tool.

The inventive bone anchor can be used in various methods relating to spinal surgery in which one or more pedicle screws are placed in one or more pedicles of one or more vertebrae. In certain embodiments, an inventive bone anchor is placed in a void in a pedicle and/or in a void in a vertebral body to receive a pedicle screw. An example of placement of a pedicle screw in a vertebra is described in the article by Y. J. Kim and L. G. Lenke entitled, “Thoracic pedicle screw placement: free-hand technique,” Neurology India, Vol. 53, pp. 512-519, December 2005, which is incorporated herein by reference. In some embodiments, a bone anchor is placed in a void in the transverse process or spinous process. In some embodiments, a bone anchor is placed in a void in the sacrum. In various aspects, a bone anchor placed in a vertebra improves the integrity of the implant site for receiving a fastening device, e.g., a pedicle screw, a fixation device, a pin, a rod, a bone screw, or the like. The fastening device can be used to secure rigid or flexible rods, pins, plates, pedicle fixation systems, or the like which may be used to stabilize and/or relocate one or plural vertebrae.

In certain embodiments, a method of using the inventive bone anchor for spinal surgery comprises (a) evaluating an implant site and (b) providing an inventive bone anchor as described herein to improve the integrity of the implant site. For example, the method can be used to improve the integrity of a placement site for implanting a pedicle fixation device in a patient. The site can be evaluated for placement of a pedicle screw or fastening device into one or more vertebrae, and the bone anchors used to improve the structural integrity of bone at the site for receiving a pedicle screw or fastening device. The method can be carried out on a patient presenting any indication selected from the following group: painful spinal instability, post-laminectomy spondylolisthesis, pseudoarthrosis, spinal stenosis, degenerative scoliosis, unstable vertebral fractures, spinal osteotomies, nerve compression, diseased bone, prior surgical intervention which needs revision, vertebral tumor or infection.

The step of evaluating the implant site can occur before surgical intervention or during surgical intervention. A physician can image or inspect directly the affected area. In some embodiments, a physician assesses characteristics of the bone into which a pedicle screw or fastening device will be placed. Preoperative imaging and assessment can be performed with radiography and CT scanning Assessed characteristics can include bone density, bone structure, bone shape, presence of bone defects at the affected area, transverse diameter of one or more pedicles, sagittal diameter of one or more pedicles, a length associated with a pedicle and vertebral body into which a pedicle screw will be placed, and quality of one or more vertebral bodies. Based upon preoperative imaging and assessment, a physician can select one or more candidate bone anchors for placement during spinal surgery. In some embodiments, the physician selects candidate bone anchors from a group of preformed and/or non-preformed bone anchors.

In certain embodiments, the step of evaluating the implant site occurs during surgical intervention. A physician can observe directly a bony defect at the implant site, which may need alteration, e.g., removal, revision, or reconstruction. In some embodiments, a physician encounters or discovers a defect after initiating a procedure for placement of a pedicle screw or fastening device, and evaluates the implant site. As an example, a bone chip or fracture may occur in the bone during decorticating the pedicle, or insertion of a pedicle screw. As another example, a pedicle probe, used to open a path or hole for a pedicle screw, can have an undesirable trajectory risking a breach of the cortex of the pedicle or vertebral body, or the pedicle probe may breach the cortex of the pedicle or vertebral body. As additional examples, the pedicle screw encounters osteoporotic bone or strips the hole into which the screw is advanced. The screw then loses it grip in the hole, and cannot provide tightening of the screw to the bone. In such and similar cases, the physician can evaluate the implant site and select an inventive bone anchor to improve the integrity of the implant site. In various embodiments, the inventive bone anchor provides a lining within the void in the bone which can grip the surrounding bone and providing a surface for the screw to tighten against.

The step of providing an inventive bone anchor to improve the integrity of the implant site comprises placing an inventive bone anchor at the implant site such that the bone anchor improves the integrity of the implant site for receiving a pedicle screw or fastening device. In various embodiments, the integrity of the implant site is improved by the inventive bone anchor when the anchor provides either or both of (1) improved gripping strength of a pedicle screw or fastening device into the implant site and (2) improved structural support of the bone anchor/bone combination for holding securely the pedicle screw or fastening device. In some embodiments, the bone anchor covers or repairs breached cortex. In some embodiments, the bone anchor allows altering of the trajectory of a pedicle probe. In some embodiments, an inventive bone anchor is placed in a prepared void in a pedicle and/or vertebral body. The pedicle and/or vertebral body can be osteoporotic, diseased, altered due to trauma, or exhibit a structural defect.

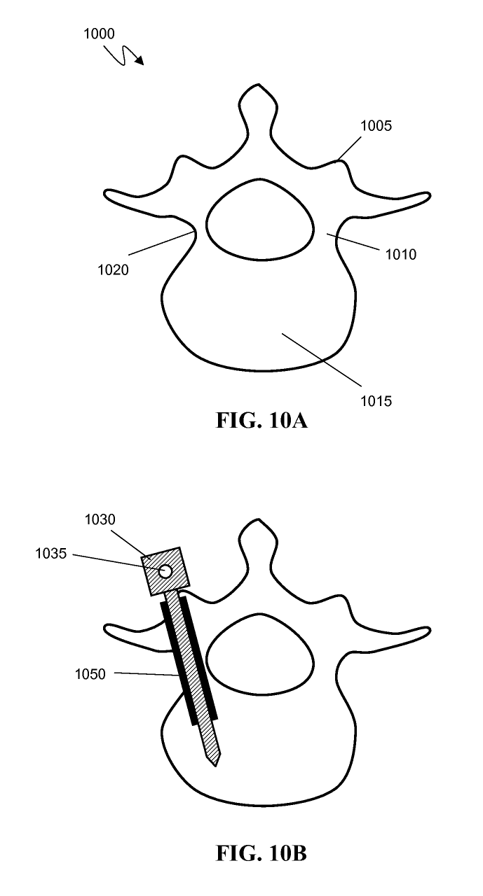

An example of placement of the inventive bone anchor in a defective pedicle is depicted in FIGS. 10A-10B. A vertebra 1000 can exhibit a defect in or defective pedicle 1020, as compared to a normally-developed pedicle 1010. In some embodiments, it is necessary to place one or two pedicle screws into the vertebra to stabilize or fixate the vertebra or one or more adjacent vertebrae. Due to the pedicle's structural defect, a pedicle screw 1030 would normally breach the pedicle's cortex and further weaken the pedicle. In certain embodiments, a void is prepared such that the bone anchor 1050 breaches a portion of the pedicle's cortex when placed, yet provides for securing of the pedicle screw 1030 to at least a portion of each of the pedicle, the vertebral body 1015 and the superior articular facet 1005. Over time, the bone anchor 1050 can subsequently be resorbed and transformed to bone, providing additional strength to the defective pedicle. A rod or pin can be secured to a hole 1035 in the head of pedicle screw 1030 to provide stabilization or fixation of one or more vertebrae.

In some embodiments, the inventive bone anchor is made malleable and pressed onto or into, or formed around a defective pedicle to improve the structural integrity of the pedicle, e.g., to repair, reconstruct, or reinforce the pedicle. For example, an abnormally thin pedicle can be surrounded with a sheath-like bone anchor which subsequently is resorbed. In some embodiments, a portion of a bone anchor is placed in a pilot hole with an undesirable trajectory in a pedicle, so that the trajectory of the pedicle screw is altered toward a more favorable trajectory. In some embodiments, a pilot hole with an undesirable trajectory is filled with the bone anchor and a new pilot hole is formed with a more favorable trajectory. In some embodiments, prior misplaced or failing pedicle screws are removed and the bone anchors inserted into the voids such that new pedicle screws can be placed and secured in the vertebra. In some embodiments, a portion of a bone anchor can be applied to a pedicle or vertebral body as a patch to improve the structural integrity of the pedicle or vertebral body. It will be appreciated that there exist a variety of ways to improve the integrity of a placement site in a pedicle, vertebral body, or other aspect of a vertebra with the inventive bone anchors.

In certain embodiments, a void is prepared in a pedicle and/or vertebral body, or other aspect of a vertebra, and a preformed bone anchor is provided to fit into the prepared void. A preformed bone anchor can have any shape as depicted in FIGS. 1-9, or similar shape. In various embodiments, the preformed bone anchor provides for secure attachment of a fastening device to the bone.

In various embodiments where the implant site is irregular in shape, an inventive bone anchor is made malleable by heating or adding a solvent, so that it can be more readily pressed into the implant site and adapt to irregularities in the native bone. The bone anchor is then substantially solidified. The anchor can be substantially solidified by the addition of an agent such as a chemical agent, addition of energy such as UV light, IR radiation, microwave radiation, or addition or dissipation of heat. In some embodiments, the anchor solidifies by allowing the implant to cool to body temperature or by allowing a solvent or plasticizer to diffuse out of the anchor material.

As discussed herein, in some embodiments, an inventive bone anchor is made from a composite including a monomer, prepolymer, or telechelic polymer that is polymerized in situ. An initiator or catalyst can be injected into the tissue site as part of the anchor placement step, before or after placement. Alternatively or in addition, an anchor can be exposed to conditions that stimulate polymerization, cross-linking and solidification after placement. In another embodiment, a lower molecular weight polymer is used to make a bone anchor, and the polymer is cross-linked and/or further polymerized following placement. Of course, if a bone/polymer composite is sufficiently malleable at body temperature, even if that is greater than the glass transition temperature, no pre-placement treatment of the anchor may be necessary.

After implantation, an inventive bone anchor typically stays at the site of implantation and is gradually transformed at least in part to host tissue by the body as bone forms in and around it. A bone anchor design is typically selected to provide the mechanical strength necessary for the implantation site. At least a portion of the anchor can be adapted to be resorbed over a period of time having any value in a range from approximately 1 month to approximately 6 years. The rate can depend on the polymer used in the bone-anchor composite, the patient's ability to develop cells of the osteoclastic and osteoblastic lineages that incorporate the implant, the site of implantation, the condition of the wound, the patient, disease condition, etc. In certain embodiments, an implanted bone anchor persists in its original form for approximately 1 month to approximately 6 months. In other embodiments, the anchor persists for approximately 6 months to approximately 1 year. In other embodiments, the anchor persists for approximately 1-2 years. In other embodiments, the anchor persists for approximately 2-3 years. In other embodiments, the anchor persists for approximately 3-5 years. During these periods, portions of the bone anchor can be resorbed.

In yet another aspect, a step of providing a bone anchor can include preparing a bone anchor by heating the bone/polymer or bone substitute/polymer composite until it becomes moldable, pliable or flowable (e.g., to a temperature value, which can be any value between approximately 40° C. and approximately 130° C.). In various embodiments, a step of heating the composite can comprise heating the material to a temperature within a range between about 40° C. and about 45° C., between about 45° C. and about 50° C., between about 50° C. and about 55 C, between about 55 C and about 60° C., between about 60° C. and about 70° C., between about 70° C. and about 80° C., between about 80° C. and about 90° C., between about 90° C. and about 100° C., between about 100° C. and about 110° C., between about 110° C. and about 120° C., and yet between about 120° C. and about 130° C. in some embodiments. Once moldable or pliable, the bone anchor can be formed by pressing it or injecting it into a mold, whereafter it becomes substantially rigid after cooling. In certain aspects, the molded anchor is heated prior to placement, so that it becomes semi-malleable, facilitating insertion into irregular-shaped voids. Once the anchor is implanted and allowed to cool to body temperature (approximately 37° C.), the composite becomes set providing a substantially rigid bone anchor.

In certain aspects, a step of providing a bone anchor can include preparing the bone anchor by combining a plurality of particles comprising an inorganic material, a bone substitute material, a bone-derived material, or combinations thereof; and a polymer (e.g., polycaprolactone, poly(lactide), poly(glycolide), poly(lactide-co-glycodide), polyurethane, etc.); and adding a solvent or pharmaceutically acceptable excipient so that the resulting composite is flowable or moldable. The flowable or moldable composite can then be placed into a two-piece mold to form an anchor of a desired shape. As the solvent or excipient diffuses out of the composite, the anchor solidifies. Advantages of molding an inventive bone anchor during a step of providing a bone anchor include flexibility in choice and design of an anchor once the implantation site becomes visible to an attending physician.

In some embodiments, a bone-anchor composite is transformed to a flowable phase-state and injected into an implantation site directly, so that the bone anchor is formed in situ. For example, a fastening-device form, representative of a fastening device, can be positioned in the implant site at its approximate intended location. The fastening-device form can be located centrally within the implant site. (See FIG. 7) The flowable composite can then be injected to fill the voids between the fastening-device form and the surrounding bone. After the composite solidifies to an extent, the fastening-device form can be removed, e.g., unscrewed, leaving a ready-to-use anchor securely formed in intimate contact with the surrounding native bone. In certain embodiments, there will be low adhesion between the material comprising the fastening-device form and the solidified composite.

In certain embodiments, an inventive bone anchor is formed at an implant site via injecting, pressing, or tamping the flowable or malleable composite into place. In some embodiments, the composite is rendered into a liquid or semi-liquid state and injected into the implant site using a 3 mm cannula. Flowable bone-anchor composite can be conveyed to the implant site via the cannula. In some embodiments, the bone anchor composite is rendered into a malleable state and pressed or tamped into the implant site, e.g., tamped into place with a bone tamp. After subsequent solidification, the composite can be adapted to retain a fastening device, e.g., drilling a hole into the composite to receive a screw, threading the hole, bonding a fastening device into an unthreaded hole, etc.

### Kits

In another aspect, the invention provides various kits for use in orthopedic or dental procedures. The kits can include at least one preformed bone anchor, or at least enough bone/polymer or bone substitute/polymer composite for the formation of one bone anchor. The kits can optionally include any of the following: fastening devices, bone-anchor molds, fastening-device forms, an anchor-insertion or placement tool or tools, one or more bone-removal tools or tools to adapt the placement site to accommodate a bone anchor, a cannula, a tool to adapt the size or shape of an anchor to fit into an implantation site, and instructions for using the tools and placing an anchor. A kit can include a tool for changing the phase-state of the bone anchor composite.

One type of kit can include at least one preformed inventive bone anchor, or pieces of a preformed anchor, and can include instructions for placing and using the anchor. In some embodiments, a kit includes a plurality of preformed anchors in similar or various sizes, for example 2, 3, 5, 10, 15, etc. anchors per kit with anchor diameters of substantially equivalent value or of various diameters of any value between about 5 millimeters and about 20 millimeters. For example, the kit can include 2 anchors having an outer diameter of about mm, two having an outer diameter of about 7.5 mm, two having an outer diameter of about 10 mm, two having an outer diameter of about 15 mm, and two having an outer diameter of 20 mm. The kits can include mixed designs, for example anchors with substantially constant inner and outer diameters and anchors with gradually varying inner and/or outer diameters. The lengths of the bone anchors provided in a kit be any value within a range from about 5 mm to about 20 mm. In some embodiments, the lengths are longer than required for expected implant sites, and the anchors cut to length with a scalpel prior to placement or after placement. The kits can include fastening devices which mate to the anchors, and can include more than one type of mating fastening device per anchor. In certain embodiments, the kits include tools for placing the bone anchor, and optionally include additional tools for inserting the fastening device. In some embodiments, a kit includes one or more tools, e.g., a reamer, drill, cutting or grinding tool, for adapting the implantation site to accommodate a bone anchor. In some embodiments, the kit includes one or more tools, e.g., a scalpel, a cutting, abrasive, or grinding tool, to adapt the bone anchor to fit within an implantation site. All components of the kit, and the kit itself, can be sterilely packaged.

Another type of kit can include a quantity of bone/polymer or bone substitute/polymer composite sufficient in amount to form at least one bone anchor, one or more anchor molds, and can include instructions for forming, placing and using the anchor. Such a kit can include a heating device, solvent, or pharmaceutically acceptable excipient for making the anchor moldable, pliable or flowable. A cannula can be provided with the kit. The kit can include mating fastening devices, and can include more than one type of mating fastening device per anchor. In some embodiments, the kits include tools for placing the bone anchor, and can include additional tools for inserting the fastening device.

Another type of kit can include a quantity of bone/polymer or bone substitute/polymer composite sufficient in amount to form at least one bone anchor, one or more fastening-device forms, an injection syringe or cannula, and can include instructions for forming, placing and using the anchor. Various amounts of the composite can be packaged in a kit, and all components of the kit, and the kit itself, can be sterilely packaged. The kit can include mating fastening devices, and can include more than one type of mating fastening device per anchor. In various embodiments, the kits can optionally include tools for placing the bone anchor, and can include additional tools for inserting the fastening device.

An inventive “salvage” kit represents an additional embodiment of an inventive bone anchor kit. In various embodiments, the salvage kit is kept in or near the operating room. The kit is used for surgical situations where a pedicle screw cannot maintain purchase with the bone inside the pedicle, e.g., the bone is osteoporotic, diseased, defective, deformed, the threaded hole becomes stripped, the pedicle screw has an undesirable trajectory, etc. The kit can provide inventive bone anchors of two or three different designs and/or different sizes, and can include preformed and non-preformed bone anchors. The salvage kit can contain at least one T-handle reamer. The reaming head can be conical in shape, or interchangeable, to allow reaming of different size holes. In some embodiments, detachable reaming heads of various sizes can be provided, each individually attachable to the T-handle's shaft. A reaming head can be selected based on the size of a void at the implant site. The kit can include a tool to insert a bone anchor, e.g., a T-handle inserter. In various embodiments, the salvage kit is for unplanned situations that arise in surgery. In circumstances where the implantation site becomes damaged during a normally routine procedure, the kit can be relied upon to salvage the procedure. For example, a defective implantation site could be reamed to a substantially round hole of a larger diameter, and a bone anchor inserted into the newly-formed hole.

All literature and similar material cited in this application, including, but not limited to, patents, patent applications, articles, books, treatises, and web pages, regardless of the format of such literature and similar materials, are expressly incorporated by reference in their entirety. In the event that one or more of the incorporated literature and similar materials differs from or contradicts this application, including but not limited to defined terms, term usage, described techniques, or the like, this application controls.

The section headings used herein are for organizational purposes only and are not to be construed as limiting the subject matter described in any way.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art.

The claims should not be read as limited to the described order or elements unless stated to that effect. It should be understood that various changes in form and detail may be made by one of ordinary skill in the art without departing from the spirit and scope of the appended claims. All embodiments that come within the spirit and scope of the following claims and equivalents thereto are claimed.

1. A bone anchor comprising:
an elongate element having a near end, a distal end, an inner surface and an outer surface, wherein the element is adapted for placement within a void in a bone and is adapted to receive and secure a fastening device; and wherein the element is formed from a composite comprising:
a plurality of particles selected from the group consisting of: particles of bone-derived material, bone particles, particles of bone substitute material, inorganic particles, and any combination thereof; and
a polymer with which the plurality of particles have been combined.

2. The bone anchor as claimed in claim 1, wherein the composite can undergo a phase transition from a formable, moldable, pliable or flowable state to a substantially solid state, and
the phase transition occurs within biocompatible temperature ranges or biocompatible chemical conditions.

3. The bone anchor as claimed in claim 2, wherein the bone anchor was transitioned to a substantially solid state after placement in the void in a bone.

4. The bone anchor as claimed in claim 1, wherein the element is tubular in shape, having at least one inner diameter and at least one outer diameter, and a wall extending the length of the element between the at least one inner diameter and the at least one outer diameter.

5. The bone anchor as claimed in claim 4, further comprising:
at least one slot incorporated into at least a portion of the anchor's wall, the at least one slot running in a direction along the length of the anchor; and wherein
insertion of the fastening device into the central core of the anchor expands the portion of the wall incorporating the slots radially outward.

6. The bone anchor as claimed in claim 4, further comprising a shape feature selected from the following group: an inner diameter substantially constant along the length of the anchor, an outer diameter substantially constant along the length of the anchor, an inner diameter gradually decreasing from the near end to the distal end, an outer diameter gradually decreasing from the near end to the distal end, and an outer diameter gradually increasing from the near end to the distal end.

7. The bone anchor as claimed in claim 4, wherein the maximum outer diameter is in a range between about 5 millimeters and about 10 millimeters, and the maximum inner diameter is in a range between about 2 millimeters and about 8 millimeters.

8. The bone anchor as claimed in claim 4, wherein the maximum outer diameter is in a range between about 10 millimeters and about 20 millimeters, and the maximum inner diameter is in a range between about 8 millimeters and about 17 millimeters.

9. The bone anchor as claimed in claim 4, wherein the length of the anchor is in a range between about 3 millimeters and about 5 millimeters.

10. The bone anchor as claimed in claim 4, wherein the length of the anchor is in a range between about 5 millimeters and about 10 millimeters.

11. The bone anchor as claimed in claim 4, wherein the length of the anchor is in a range between about 10 millimeters and about 20 millimeters.

12. The bone anchor as claimed in claim 4, wherein the anchor incorporates a feature selected from the group consisting of: a smooth outer surface, a threaded outer surface, a ridged outer surface, a ribbed outer surface, an outer surface having protrusions, an outer surface having indentations, a grooved outer surface, and any combination thereof.

13. The bone anchor as claimed in claim 4, wherein the anchor incorporates a feature selected from the group consisting of: a smooth inner surface, a threaded inner surface, a ridged inner surface, a ribbed inner surface, an inner surface having protrusions, an inner surface having indentations, a grooved inner surface, and any combinations thereof.

14. The bone anchor as claimed in claim 4, wherein the anchor incorporates a plurality of inner diameters along the length of the anchor, each inner diameter corresponding to a portion of the length of the anchor, and at least one portion located at the distal end having a threaded inner surface; wherein
a fastening device engages the threaded inner surface at the distal end and compresses the bone anchor along its length upon tightening the fastening device, the compressing action causing the walls along a portion of the bone anchor to expand radially outward.

15. The bone anchor as claimed in claim 4, wherein the anchor incorporates a feature at its near end selected from the group consisting of: a flanged head, a pan head, a slotted head, a socket head, a hexagonal head, and a square head.

16. The bone anchor as claimed in claim 4 adapted to receive a bayonet fastening device, wherein the bayonet fastening device can be rotated to a locking position upon insertion.

17. The bone anchor as claimed in claim 4 adapted to receive a latching rivet-like fastening device, wherein the rivet-like fastening device can be tapped, pressed or driven into a locked position.

18. The bone anchor as claimed in claim 4, wherein the fastening device is a device selected from the group consisting of: pedicle screw, screw, bolt, pin, post, rod, and spring pin.

19. The bone anchor as claimed in claim 4, wherein the fastening device is a device selected from the group consisting of: cancellous, cortical, and malleolar screws.

20. The bone anchor as claimed in claim 1, wherein the polymer comprises a material selected from the group consisting of: polylactides, polyglycolides, starch poly(caprolactone), poly(caprolactones), poly(L-lactide), poly(lactide-co-glycolide), poly(D,L-lactide-co-glycolide), poly(L-lactide-co-glycolide), poly(L-lactide-co-D,L-lactide), polyurethanes, polycarbonates, polyarylates, poly(propylene fumarates), polyphosphazenes, polymethylmethacrylates, polyacrylates, polyesters, polyethers, stereoisomers of the above, co-polymers of the above, lactide-glycolide copolymers, polyglyconate, poly(anhydrides), poly(hydroxy acids), poly(alkylene oxides), poly(propylene glycol-co fumaric acid), polyamides, polyureas, polyamines, polyamino acids, polyacetals, poly(orthoesters), poly(pyrolic acid), poly(glaxanone), poly(phosphazenes), poly(organophosphazene), poly(dioxanones), polyhydroxybutyrate, polyhydroxyvalyrate, polyhydroxybutyrate/valerate copolymers, poly(vinyl pyrrolidone), polycyanoacrylates, glucose-based polyurethanes, lysine-based polyurethanes, polysaccharides, chitin, starches, celluloses, PEGylated-poly(lactide-co-glycolide, PEGylated-poly(lactide), PEGylated-poly(glycolide), collagen, polysaccharides, agarose, glycosaminoglycans, alginate, chitosan, tyrosine-based polymers, polypyrrole, polyanilines, polythiophene, polystyrene, non-biodegradable polyesters, non-biodegradable polyureas, poly(vinyl alcohol), non-biodegradable polyamides, poly(tetrafluoroethylene), expanded polytetrafluoroethylene (ePTFE), poly(ethylene vinyl acetate), polypropylene, non-biodegradable polyacrylate, non-biodegradable polycyanoacrylates, non-biodegradable polyurethanes, copolymers of poly(ethyl methacrylate) with tetrahydrofurfuryl methacrylate, polymethacrylate, non-biodegradable poly(methyl methacrylate), polyethylene (including ultra high molecular weight polyethylene (UHMWPE)), polypyrrole, polyanilines, polythiophene, poly(ethylene oxide), poly(ethylene oxide co-butylene terephthalate), poly ether-ether ketones (PEEK), polyetherketoneketones (PEKK), and combinations thereof.

21. The bone anchor as claimed in claim 1, wherein the particles comprise from about 50% to about 70% by weight of the composite from which the bone anchor is formed.

22. The bone anchor as claimed in claim 1, wherein the particles comprise about 63% by weight of the composite from which the bone anchor is formed.

23. The bone anchor as claimed in claim 1, wherein the composite forming the bone anchor is osteoinductive or osteoconductive.

24. The bone anchor as claimed in claim 1, wherein the bone anchor is placed in a void in a vertebra or in the sacrum.

25. The bone anchor as claimed in claim 1, wherein the bone anchor is placed in a void in the pedicle of a vertebra or the body of a vertebra.

26. The bone anchor as claimed in claim 1, wherein the bone anchor is adapted to be resorbed over a period from about 1 month to about 6 months.

27. The bone anchor as claimed in claim 1, wherein the bone anchor is adapted to be resorbed over a period from about 6 months to about 1 year.

28. The bone anchor as claimed in claim 1, wherein the bone anchor is adapted to be resorbed over a period from about 1 year to about 2 years.

29. The bone anchor as claimed in claim 1, wherein the bone anchor is adapted to be resorbed over a period from about 2 years to about 3 years.

30. The bone anchor as claimed in claim 1, wherein the bone anchor is adapted to be resorbed over a period from about 3 years to about 5 years.

31. The bone anchor as claimed in claim 1, wherein the composite can undergo a reversible phase transition from a formable, moldable, pliable or flowable state to a substantially solid state; and
the phase transition occurs within a temperature range selected from the group consisting of: between about 40° C. and about 45° C., between about 45° C. and about 50° C., between about 50° C. and about 55° C., between about 55° C. and about 60° C., between about 60° C. and about 70° C., between about 70° C. and about 80° C., between about 80° C. and about 90° C., between about 90° C. and about 100° C., between about 100° C. and about 110° C., between about 110° C. and about 120° C., and between about 120° C. and about 130° C.

32. A bone anchor for spinal surgery comprising:
a substantially cylindrical, conical or tulip shaped elongate element adapted for placement in a void in the pedicle of a vertebra of a subject, the elongate element further adapted to receive and secure a fastening device; wherein the elongate element is formed from a composite comprising:
bone particles; and
a polymer; and wherein
at least a portion of the bone anchor expands radially outward upon insertion of a fastening device into the elongate element.

33. (canceled)

34. A method of forming a bone anchor in vivo, the method comprising:
placing a fastening-device form into a void in a bone;
injecting a flowable composite into the vacancy between the fastening-device form and the surrounding bone, the composite comprising a plurality of particles of a bone substitute material, bone-derived material, bone particles, inorganic material, or any combination thereof combined with a polymer;
transforming the composite to a substantially solid state; and
removing the fastening-device form.

35. The method of claim 34, wherein the injecting comprises injecting the anchor into a void of a vertebra or sacrum.

36. A method of placing the bone anchor of claim 1, the method comprising:
implanting the bone anchor into a void in the pedicle or the body of a vertebra or the sacrum of a subject; and
securing a fastening device into the bone anchor.

37. The method of claim 36, wherein the implanting is repeated for multiple vertebrae of a subject.

38. The method of claim 36, wherein the implanting comprises molding or adapting the shape of the anchor for conformity with a void in a vertebra or sacrum.

39. The method of claim 36, wherein the implanting comprises sequentially placing pieces of the anchor into a void in a vertebra or sacrum.

40. A method of placing a bone anchor, the method comprising:
rendering a composite into a flowable state, the composite comprising (1) a plurality of particles of an inorganic material, a bone-substitute material, a bone-derived material, bone particles, or any combination thereof; and (2) a polymer;
injecting the composite into a void within a bone; and
forming a hole in the composite bone anchor to receive a fastening device

41. (canceled)

42. (canceled)

43. (canceled)

44. (canceled)

45. (canceled)

46. (canceled)

47. A method of placing a bone anchor in a vertebra, the method comprising:
evaluating a characteristic of at least a portion of the vertebra;
selecting a type of bone anchor based upon the evaluated characteristics;
preparing a site in the vertebra to receive the bone anchor; and
providing the bone anchor to the prepaired site.

48. The method of claim 47, wherein the evaluated characteristic comprises bone density, bone disease, bone structure, or bone defect.

49. The method of claim 47, wherein the portion of the vertebra comprises a pedicle.

50. The method of claim 47, wherein the portion of the vertebra comprises the vertebral body.

51. The method of claim 47, wherein the selected type of bone anchor comprises an anchor structure preformed from bone/polymer or bone substitute/polymer composite.

52. The method of claim 47, wherein the selected bype of bone anchor comprises a moldable anchor formed from bone/polymer or bone substitute/polymer composite.

53. The method of claim 47, wherein the step of preparing the site comprises forming a void in the site.

54. The method of claim 47, wherein the step of preparing the site comprises reaming, drilling, grinding, cutting, or threading bone at the site.

55. The method of claim 47, wherein the step of preparing the site comprises revising prior surgical intervention at the site.

56. The method of claim 47, wherein the step of providing the bone anchor comprises inserting or affixing the bone anchor at the site.

57. The method of claim 47, wherein the step of providing the bone anchor comprises pressing or tamping the bone anchor into the site.

58. The method of claim 47, further comprising reaming, drilling, cutting, grinding, or threading the bone anchor placed at the site.

59. A bone anchor formed from a composite comprising:
a plurality of particles selected from the group consisting of: particles of bone-derived material, bone particles, particles of bone substitute material, inorganic particles, and any combination thereof; and
a polymer with which the plurality of particles have been combined.

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)
